(19) United States
(12) Patent Application Publication
(10) Pub. No.: US 2010/0129933 A1
(43) Pub. Date: May 27, 2010

(54) METHOD FOR DETECTING THE BINDING BETWEEN MDM2 AND THE PROTEASOME

(75) Inventors: Janine Arts, Breda (NL); Christine Blattner, Waghaeusel (DE); Roman Nikolaevich Kulikov, Stutensee (DE); Annemie Francine Valckx, Lille (BE)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: Forschungszentrum Karlsruhe GmbH

(21) Appl. No.: 12/597,356

(22) PCT Filed: Apr. 26, 2007
(86) PCT No.: PCT/EP08/55088

§ 371 Date: Oct. 23, 2009

(30) Foreign Application Priority Data
Apr. 26, 2007 (EP) 07107042.9

(51) Int. Cl. G01N 33/53; C12N 15/12; C07K 2/00

(52) U.S. Cl. 436/501; 536/23.5; 530/300

(57) ABSTRACT

This invention provides both nucleic acid and amino acid sequences encoding for isolated protein-HDM2 binding sites as well as for isolated protein-proteasome binding sites (the ED(X)Y sequences). In a further aspect this invention also provides related nucleic acids, amino acids, vectors, host cells, pharmaceutical compositions and articles of manufacture. This invention further provides methods for determining whether a test compound interacts with the binding between HDM2 and the proteasome, as well as pharmaceutical compositions comprising said test compound, in particular as anti-cancer agent, even more particular for treating cell proliferative disorders in a subject.

This invention is based on the characterization of the interaction of HDM2 or related proteins with the proteasome and how disturbance of this interaction can affect e.g. the ubiquitinin-(Ub)-proteasome proteolysis (UPS) pathway. It accordingly provides both nucleic acid and amino acid sequences encoding for isolated protein-HDM2 binding sites, as well as for isolated protein-proteasome binding sites (the ED(X)Y sequences and their counterparts). In a further aspect this invention also provides related nucleic acids, amino acids, vectors, host cells, pharmaceutical compositions and articles of manufacture. This invention further provides methods for determining whether a test compound interacts with the binding between HDM2 and the proteasome.

### BACKGROUND OF THE INVENTION

Hdm2 is a key oncogene which is activated in a large number of cancer patients through various mechanisms including hdm2 gene amplifications, and deletion of upstream tumor supressors such as p14ARF and PTEN. Hdm2 is overexpressed in several types of malignancies including osteosarcomas, soft tissue sarcomas and gliomas and high levels of hdm2 are associated with poor prognosis.¹ Interestingly, a single nucleotide polymorphism in the hdm2 promoter which increases hdm2 expression has been associated with accelerated tumor formation in both hereditary and sporadic cancers in humans².

HDM2 promotes tumorigenesis by associating with cell cycle regulatory proteins, modulating their activity and stability. The number of HDM2 substrates is rapidly expanding, key examples include the tumor suppressor p53 and its family members p63 and p73, E2F1 and p21^{waf1,cip1}.³ Most extensively studied is p53. HDM2 binds and ubiquitinates the p53 protein which results in a rapid degradation of p53 by the proteasome. Abrogation of HDM2-p53 complex degradation causes p53 stabilization and subsequent transcriptional activation of p53 downstream genes (reviewed in Brooks and Gu⁴). In addition to the ubiquitin ligase function, other activities of HDM2 are also required for p53 degradation, as evidenced by the accumulation of ubiquitylated p53 when phosphorylation in the central domain of HDM2 is abrogated (Blattner et al., 2002⁵). The association of HDM2 with different subunits of the 26S proteasome such as S4, S5a, S6a and S6b (3^{rd} Mdm2 workshop, September 2005 in Constance, Germany) might play a key role in this process.

Consistent with the key role of HDM2 tumorigenesis, antagonists of the hdm2 oncogene, i.e., peptides and small molecules, inhibit tumor cell proliferation in vitro and the growth of human xenografts in immunodeficient mice in vivo^{6,7}. Hdm2 antagonists might even exhibit anti-proliferative effects in tumour cells that are devoid of functional p53. This positions the HDM2 protein as an attractive target for the development of anti-cancer therapy.

Given the central role of HDM2 in tumorigenesis, it is to be expected that modulation of the HDM2-proteasome interaction could provide a novel mechanism for the development of new chemotherapeutics for the treatment of cancer.

It is an object of the present invention to characterize protein-HDM2 binding sites, as well as protein-proteasome binding sites (the ED(X)Y sequences and their counterparts), that allow to identify compounds capable to interfere with e.g. the HDM2—proteasome interaction and accordingly useful in the treatment of cell proliferative disorders e.g. through disturbance of the UPS-pathway.

### REFERENCES

1. Cordon-Cardo C, Latres E, Drobnjak M, Oliva M R, Pollack D, Woodruff J M, Marechal V, Chen J, Brennan M F and Levine A J. Molecular abnormalities of mdm2 and p53 genes in adult soft tissue sarcomas. (1994) Cancer Res. 54, 794-9.
2. Bond G L, Hu W W, Bond E E, Robins H, Lutzker S G, Arva N C, Bargonetti J, Bartel F, Talbert H, Wuerl P, Onel K, Yip L, Hwang S J, Strong L C, Lozano G and Levine A J. A single nucleotide polymorphism in the MDM2 promoter attenuates the p53 tumor suppressor pathway and accelerates tumor formation in humans. (2004) Cell 119, 591-602.
3. Zhang Z and Zhang R. p53-independent activities of Mdm2 and their relevance to cancer therapy. Current Cancer Drug Targets; 2005; 5: 9-20.
4. Brooks C L and Gu W. p53 ubiquitination: mdm2 and beyond. Molecular Cell 21, 307-315.5.
5. Blattner C, Hay T, Meek D, Lane D P. Hypophosphorylation of Mdm2 augments p53 stability. (2002) Mol. Cell. Biol., 22, 6170-6182,
6. Vassilev L T, Vu B T, Graves B, Carvajal D, Podlaski F, Filipovic Z, Kong N, Kammlott U, Lukacs C, Klein C, Fotouhi N, Liu E A. In Vivo Activation of the p53 Pathway by Small-Molecule Antagonists of MDM2. Science; 2004; 303: 844-848.
7. Grasberger B L, Lu T, Schubert C, Parks D J, Carver T E, Koblish H K, Cummings M D, LaFrance L V, Milkiewicz K L, Calvo R R, Maguire D, Lattanze J, Franks C F, Zhao S, Ramachandren K, Bylebyl G R, Zhang M, Manthey C L, Petrella E C, Pantoliano M W, Deckman, I C, Spurlino, J C, Maroney A C, Tomczuk B E, Molloy C J, Bone R F. Discovery and Cocrystal Structure of Benzodiazepinedione HDM2 Antagonists that Activate p53 in Cells. J. Med. Chem.; 2005; 48: 909-12.

### SUMMARY OF THE INVENTION

The present invention provides assays that make use of the interaction of HDM2, related proteins or protein binding fragments thereof with proteins or small molecules, as well as of protein-proteasome binding sites, in particular interactions comprising a proteasome subunit selected from the group consisting of S2, S4, S5a, S6a or S6b or a fragment thereof.

The assays are useful to identify whether a test compound can alter the interaction of HDM2, a related protein or a protein binding fragment thereof, with another protein, the proteasome or a proteasome subunit. The assays are also useful to determine whether the test compound is an agonist or antagonist of the UPS-pathway. The above assays can be performed in a variety of formats including competitive, non-competitive and comparative assays in which the interaction of HDM2 (SEQ ID NO:5), related proteins or protein binding fragments thereof, with another protein, the proteasome or proteasome subunit is assessed as a positive or negative control or compared to the results obtained with the test compound.

In another aspect the present invention relates to the isolated and purified polypeptide and polynucleotide molecules encoding for isolated protein-HDM2 binding sites as well as for isolated protein-proteasome binding sites (the ED(X)Y sequences), and the use of said binding regions in the assays according to the invention.

In a further embodiment the present invention relates to pharmaceutical compositions comprising the peptides, peptide mimetics or polynucleotides provided by the invention and the therapeutic use thereof to inhibit proliferative conditions, such as cancer and psoriasis. This invention provides a method for inhibiting the abnormal growth of cells, including transformed cells, by administering an effective amount of the peptides, peptide mimetics or polynucleotides of the invention. Abnormal growth of cells refers to cell growth independent of normal regulatory mechanisms (e.g. loss of contact inhibition). This includes the inhibition of tumour growth both directly by causing growth arrest, terminal differentiation and/or apoptosis of cancer cells, and indirectly, by inhibiting neovascularization of tumours.

This and further aspects of the present invention will be discussed in more detail hereinafter.

### BRIEF DESCRIPTION OF THE DRAWING

FIG. 1: JNJ-#1 binds to HDM2.

GST-HDM2, GST-HDMX were incubated with 10 μM of JNJ-#1 or with DMSO for control. Then 40 ng trypsin were added and the mixture was incubated for 15 min on ice. Samples were separated by a 12 or 15% SDS-PAGE gel and blotted onto Immobilon-P membrane. HDM2 was detected by incubating the membrane with the HDM2 antibodies 4B2, SMP14 and C18. Western blots were developed by ECL.

FIG. 2: Dose dependency of the inhibition of proteolysis of HDM2 by JNJ-#1. GST-HDM2 was incubated with the indicated doses of JNJ-#1 or with DMSO, 10 μM of active and inactive nutlin or MG132 for control. 40 ng of trypsin were added and the mixture was incubated for 15 min on ice. Samples were separated by 10% SDS-PAGE gel and blotted onto Immobilon P membrane. The membrane was incubated with the anti-HDM2 antibody 4B2. The Western blot was developed by ECL.

FIG. 3: JNJ-#1 Induces p53 and Downstream Effectors.

U-87 MG glioblastoma cells were incubated with the indicated concentrations of JNJ-#1 for 24 h. JNJ-#1 was dissolved as 5 mM stock solution in dimethylsulfoxide (DMSO) and subsequently diluted into tissue culture media to result in the final concentrations indicated. Total cell lysates were prepared and analyzed by sodium dodeclysulphate polyacrylamide gel electrophoresis (SDS/PAGE). Protein expression was detected using specific antibodies. Actin protein levels were revealed as a control for equal loading.

FIG. 4: JNJ-#1 enhances HDM2-p53 association.

JAR choriocarcinoma cells were incubated with the indicated concentrations of JNJ-#1, Nutlin-3 or the inactive enantiomer of Nutlin-3 for 1.5 hours. HDM2/p53 complexes were co-immunoprecipitated from cell lysates, and protein expression was detected using specific antibodies as indicated in the methods section. Immunoprecipitated HDM2 proteins were revealed using SMP-14 antibody (sc-965) and p53 protein was revealed as specified under Western Blot analysis.

FIG. 5 JNJ-#1 does not inhibit p53 ubiquitination in cells.

U2OS cells were transfected with His-tagged ubiquitin and incubated with 10 μM of JNJ-#1 or Nutlin-3 for 2 hours. After incubation ubiquitylated proteins were purified by adsorption to Ni2+-agarose and separated by SDS-PAGE. P53 was detected by Western blotting. (TCL: Total cell lysate)

FIG. 6: Dose dependency of the inhibition of the interaction of HDM2 and the proteasome.

GST-HDM2 was expressed in bacteria. 100 ng of the protein were incubated with proteasomes in the presence of the indicated doses of JNJ-#1 or in the presence of DMSO for control. For Input control, 10 μl of the mixture were separated by an 10% SDS-PAGE gel and blotted onto an Immobilon membrane. HDM2 was immunoprecipitated with the anti-HDM2 antibody C18 and loaded onto a 10% SDS-PAGE gel. The proteins were blotted onto Immobilon-P membranes. The top part of the membranes were hybridised with the anti-HDM2 antibody 4B2, the lower part with an antibody directed against the proteasomal subunit S8. Western blots were developed by ECL.

FIG. 7: JNJ-#1 prevents the association of HDM2 with the proteasome in living cells.

A) 293T cells were incubated for 1.5 hours with 10 μM nutlin or JNJ-#1, or with DMSO for control. Cells were lysed and HDM2 was immunoprecipitated using the C-18 antibody. The immunprecipitates were separated by a 10% SDS-PAGE gel and blotted onto Immobilon-P membrane. For expression control, 50 μg of total cell lysate (TCL) were separated by SDS-PAGE and blotted onto Immobilon-P membrane. Both membranes were hybridised with antibodies directed against S6b and HDM2. Western blots were developed by ECL. B) 293T cells were transfected with Myc-MDM2. 36 hours after transfection, 10 μM JNJ-#1 DMSO, for control, were added. Cells were lysed after 1.5 hours and MDM2 was immunoprecipitated with the anti-Myc antibody 9E10 and processed as described in part A.

FIG. 8: JNJ-#1 prevents p53 degradation in vitro.

p53 and MDM2 expressed in baculoviruses were purified and incubated for 5 hours with ubiquitin; E1 and E2 enzymes, 26S proteasomes and where indicated with the indicated doses of JNJ-#1 or with DMSO, 10 μM of active and inactive nutlin or MG132 for control. The mixture was separated by a 10% SDS-PAGE gel and blotted onto an Immobilon membrane. The membrane was hybridised with the anti-p53 antibody DO-1. The Western Blot was developed by ECL.

FIG. 9: Alignment of sequences containing the ED(X)Y-motif of different proteasomal subunits and of HDM2.

FIG. 10: HDM2 binds with the EDY sequence of the S6b protein of the 26S proteasome.

100 ng of MDM2 expressed from Baculoviruses were incubated with 100 ng of a GST-fusion protein containing the EDY sequence of the S6b protein. 10% of the sample were loaded onto a 10% SDS-PAGE gel for input control (Input). To the remaining lysate, glutathione-sepharose was added, the GST-fusion proteins were collected by centrifugation, loaded onto a 10% SDS-PAGE gel and transferred onto Immobilone P blotting membrane. The membrane was hybridised with antibodies against MDM2 and GST (Pulldown).

FIG. 11: Overexpression of the EDY sequence prevents p53 degradation.

A) H1299 cells were transfected with cDNAs encoding p53 and thioredoxin (lane 1), with cDNAs encoding p53, thioredoxin and MDM2 (lane 2), with cDNAs encoding p53, thioredoxin with a sequence from the S6b protein containing the EDY motif inserted and MDM2 (lane 3) or with cDNAs encoding p53, thioredoxin with a sequence from the HDM2 protein containing the EDY motif inserted and MDM2 (lane 4). B) U2OS cells were transfected with a cDNA encoding thioredoxin (lane 1), with a cDNA encoding thioredoxin with a sequence from the HDM2 protein containing the EDY motif inserted (lane 2) or with a cDNA encoding thioredoxin with a sequence from the S6b protein containing the EDY motif inserted (lane 3). Cells were lysed 48 hours after transfection and p53 protein levels (and PCNA levels for loading control) were determined by Western blotting.

FIG. 12: Full protein sequences containing the ED(X)Y-motif of different proteasomal subunits and of HDM2 and the polynucleotide sequences of the ED(X)Y-motifs.

FIG. 13: The central domain of HDM2 reduces the association of MDM2 with S6b.

293 cells were transfected with plasmids encoding Myc-tagged wild type (wt) or mutant MDM2 harboring the indicated deletions together with a plasmid encoding V5-tagged S6b. 24 hours after transfection, cells were lysed and MDM2 was precipitated using the antibody 9E10 coupled to protein A Agarose. The beads were washed and loaded onto a 10% SDS-PAGE gel. Proteins were transferred onto Immobilone membrane and probed for the presence of MDM2 and S6b (IP: a-Myc). TCL: 50 μg of cellular protein were separated by SDS-PAGE, blotted onto Immobilone membrane and probed for S6b and PCNA, for loading control.

### DETAILED DESCRIPTION

### Definitions

As used in this application, except as otherwise expressly provided herein, each of the following terms shall have the meaning set forth below.

“HDM2” also known as “MDM2” shall mean the “mouse double minute 2 homolog” (SwissProt entry Q00987) and is not restricted to the human protein but includes related proteins such as the mouse protein (SwissProt entry P23804), the dog protein (SwissProt entry P56950), the horse protein (SwissProt entry P56951), the cat protein (SwissProt entry Q7YRZ8) or a protein having at least 70, 80, 90, 95, 97 or 99% sequence identity to the human sequence (SwissProt entry Q00987). HDM2, also known as p53-binding protein MDM2 was originally cloned by Oliner et al. (Nature, (1992); 358:80-83). With “related proteins” is meant proteins having at least 40, 60, or 69% sequence identity to the human sequence (SwissProt entry Q00987).

“Proteasome” shall mean a large-multisubunit complex that targets the degradation of ubiquitinilated proteins.

“Proteasome subunit” shall mean proteasome subunit 6A, S6A or PSMC3; proteasome subunit 6B, S6B or PSMC4; proteasome subunit 5A, S5A or PSMD4; proteasome subunit 2, S2 or PSMD2; or proteasome subunit 4, S4 or PSMC1. It is not restricted to the human proteins but includes related proteins such as the mouse S6A (SwissProt entry Q88685), the rat S6A (SwissProt entry Q63569), the mouse S6B (SwissProt entry P54775), the rat S6B (SwissProt entry Q63570), the bovine S6B (SwissProt entry Q3T030), the macaque S6B (SwissProt entry Q4R7L3), the mouse S5A (SwissProt entry Q35226), the bovine S5A (SwissProt entry Q58DA0), the mouse S2 (SwissProt entry Q8VDM4), the bovine S2 (SwissProt entry P56701), the mouse S4 (SwissProt entry P62192), or the rat S4 (SwissProt entryP62193). It also includes a protein having at least 70, 80, 90, 95, 97 or 99% sequence identity to the human sequences S6A (SwissProt entry P17980), S6B (SwissProt entry P43686), S5A (SwissProt entry P55036), S2 (SwissProt entry Q13200), or S4 (SwissProt entry P62191).

“Administering” shall mean delivering in a manner, which is effected or performed using any of the various methods and delivery systems known to those skilled in the art. Administering can be performed, for example, topically, intravenously, pericardially, orally, via implant, transmucosally, transdermally, intramuscularly, subcutaneously, intraperitoneally, intrathecally, intralymphatically, intralesionally, or epidurally. Administering can also be performed, for example, once, a plurality of times, and/or over one or more extended periods.

“Host cells” include, but are not limited to, bacterial cells, yeast cells, fungal cells, insect cells, and mammalian cells. Bacterial cells can be transfected by methods well-known in the art such as calcium phosphate precipitation, electroporation and microinjection.

The terms “nucleic acid” and “polynucleotide” are used interchangeably herein, and each refers to a polymer of deoxyribonucleotides and/or ribonucleotides. The deoxyribonucleotides and ribonucleotides can be naturally occurring or synthetic analogues thereof.

The term “physiological conditions” shall mean, with respect to a given cell, such conditions, which would normally constitute the cell's biochemical milieu. The cell's biochemical milieu includes, without limitation some or all the proteases to which the cell is normally exposed. Such conditions include, but are not limited, to in vivo conditions.

The terms “polypeptide,” “peptide” and “protein” are used interchangeably herein, and each means a polymer of amino acid residues. The amino acid residues can be naturally occurring or chemical analogues thereof. Polypeptides, peptides and proteins can also include modifications such as glycosylation, lipid attachment, sulfation, hydroxylation, and ADP-ribosylation.

“Subject” shall mean any animal, such as a mammal or a bird, including, without limitation, a cow, a horse, a sheep, a pig, a dog, a cat, a rodent such as a mouse or rat, a turkey, a chicken and a primate. In the preferred embodiment, the subject is a human being.

“Treating” shall include, without limitation, eliminating, reversing the course of, slowing the progression of, reducing the symptoms of, or otherwise ameliorating, a disease in a subject.

“Vector” shall mean any nucleic acid vector known in the art. Such vectors include, but are not limited to, plasmid vectors, cosmid vectors, and bacteriophage vectors.

As used herein, a “compound” is an organic or inorganic assembly of atoms of any size, and includes small molecules (less than about 2500 Daltons) or larger molecules, e.g. peptides, polypeptides, whole proteins and polynucleotides.

The terms “candidate substance” and “test compound” are used interchangeably and refer to a substance that is believed to interact with the binding of HDM2, related proteins or fragments thereof, with the proteasome, proteasome subunits or fragments thereof. Exemplary candidate substances that can be investigated using the methods of the present invention include, but are not restricted to peptides, enzymes, enzyme substrates, co-factors, sugars, oligonucleotides, chemical compounds small molecules and monoclonal antibodies.

“Modulate” shall mean an increase, decrease or other alteration of any or all chemical and biological activities or properties of a wild type or mutant HDM2, proteasome, proteasome subunit or related proteins.

“Interact” shall mean detectable direct and indirect interactions between molecules, including “binding” interactions between molecules. Interactions can, for example, be protein-protein or protein-nucleic acid in nature. Such interactions can be detected using art know procedures, for example, yeast two-hybrid assay, immunoprecipitation, SPA-assay or filter binding assays.

With “proteasome binding domain” or “proteasome binding fragment” is meant part of the HDM2 protein or related protein that can bind to the proteasome or a proteasome subunit.

With “the ED(X)Y sequences and their counterparts” are meant the polypeptides as described hereinbelow and their homologues and analogues present in Hdm2, Hdm2 related proteins, the proteasome and the proteasome subunits and their counterpart polypeptides to which these sequences bind.

As used herein, “isolated” refers to the fact that the polynucleotides, proteins and polypeptides, or respective fragments thereof in question, have been removed from their in vivo environment so that they can be manipulated by the skilled artisan, such as but not limited to sequencing, restriction digestion, site-directed mutagenesis, and subcloning into expression vectors for a nucleic acid fragment as well as obtaining the protein or protein fragments in quantities that afford the opportunity to generate polyclonal antibodies, monoclonal antibodies, amino acid sequencing, and peptide digestion. In other words “isolated” indicates that a naturally occurring sequence has been removed from its normal cellular context. Thus, the sequence may be in a cell-free solution or placed in a different cellular environment or nucleic acid context. Therefore, the nucleic acids claimed herein can be present as heterologous material in whole cells or in cell lysates or in a partially, substantially or wholy purified form.

A polynucleotide is considered “purified” when it is purified away from environmental contaminants. Thus a polynucleotide isolated from cells is considered to be substantially purified when purified from cellular components by standard methods while a chemically synthesized nucleic acid sequence is considered to be substantially purified when purified from its chemical precursors. A “substantially pure” protein or nucleic acid will typically comprise at least 85% of a sample with greater percentages being preferred. One method for determining the purity of a protein or nucleic acid molecule, is by electrophoresing a preparation in a matrix such as polyacrylamide or agarose. Purity is evidenced by the appearance of a single band after staining. Other methods for assessing purity include chromatography, mass spectrometry and analytical centrifugation.

The terms “complementary” or “complementarity” as used herein refer to the capacity of purine and pyrimidine nucleotides to associate through hydrogen bonding to form double-stranded nucleic acid molecules. The following base pairs are related by complementarity: guanine and cytosine; adenine and thymine; and adenine and uracil. As used herein “complementary” means that the aforementioned relationship applies to substantially all base pairs comprising two single-stranded nucleic acid molecules over the entire length of said molecules. “Partially complementary” refers to the aforementioned relationship in which one of the two single-stranded nucleic acid molecules is shorter in length than the other such that a portion of one of the molecules remains single-stranded.

The term “hybridization” as used herein refers to a process in which a single-stranded nucleic acid molecule joints with a complementary strand through nucleotide base pairing.

The term “stringency” refers to hybridization conditions. High stringency conditions disfavor non-homologous base pairing. Low stringency conditions have the opposite effect. Stringency may be altered, for example, by temperature and salt concentration. “Stringent conditions” refers to an overnight incubation at 42° C. in a solution comprising 50% formamide, 5×SSC (750 mM NaCl, 75 mM sodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at about 65° C. Further suitable hybridization conditions are described in the examples.

The term “vector” refers to any carrier of exogenous DNA that is useful for transferring the DNA into a host cell for replication and/or appropriate expression of the exogenous DNA by the host cell.

Throughout this description the terms “standard methods”, “standard protocols” and “standard procedures”, when used in the context of molecular biology techniques, are to be understood as protocols and procedures found in an ordinary laboratory manual such as: Current Protocols in Molecular Biology, editors F. Ausubel et al., John Wiley and Sons, Inc. 1994, or Sambrook, J., Fritsch, E. F. and Maniatis, T., Molecular Cloning: A laboratory manual, 2nd Ed., Cold Spring Harbor Laboratory Press, Cold Spring Harbor, N.Y. 1989.

### EMBODIMENTS OF THE INVENTION

### HDM2 Proteins, Related Proteins and Protein Binding Fragments Thereof

This invention provides for isolated polypeptide molecules encoding protein binding fragments of HDM2 said fragments comprising at least 10 amino acids that are contiguous in the parent protein, but may desirably contain at least 11, 12, 13, 14, 15, 20, 30, 40, 60, 80, 100, 150, 200, 250, 300, or 350 amino acids that are contiguous in the parent protein and wherein said fragments are capable of binding other proteins such as but not limited to another part of the HDM2 protein, the proteasome or a proteasome subunit.

This invention further provides for isolated polypeptide molecules encoding protein binding fragments of HDM2 related proteins comprising at least 10 amino acids that are contiguous in the parent protein, but may desirably contain at least 11, 12, 13, 14, 15, 20, 30, 40, 60, 80, 100, 150, 200, 250, 300, or 350 amino acids that are contiguous in the parent protein and wherein said fragments are capable of binding other proteins.

In a particular embodiment said protein binding fragment is selected from a group of polypeptide sequences or a member of a group of polypeptide sequences comprising:

a) the N-terminal domain of HDM2 (SEQ ID NO:5),
b) amino acids 0-200 of HDM2 (SEQ ID NO:5),
c) the central domain of HDM2 (SEQ ID NO:5),
d) amino acids 200-400 of HDM2 (SEQ ID NO:5),
e) amino acids 200-300 of HDM2 (SEQ ID NO:5), or
f) amino acids 300-400 of HDM2 (SEQ ID NO:5).

In a more particular embodiment said protein binding fragment is selected from a group of polypeptide sequences or a member of a group of polypeptide sequences comprising:

a) one or two EDY sequences (SEQ ID NO:11 or SEQ ID NO 12) or homologs thereof wherein said homologs have at least 70, 80, 85, 90, 95, 97, 98 or 99% identity to SEQ ID NO:11 or SEQ ID NO 12,
b) the amino acids 252-264 (SEQ ID No:11) or 387-399 (SEQ ID No:12) of HDM2 or homologs thereof wherein said homologs have at least 70, 80, 85, 90, 95, 97, 98 or 99% identity to SEQ ID NO:11 or SEQ ID NO:12, or
c) the amino acids 257-259 or 392-394 of HDM2 (SEQ ID NO:5) or homologs thereof wherein said homologs have at least 70, 80, 85, 90, 95, 97, 98 or 99% sequence identity to SEQ ID No:5.

In an even more particular embodiment said protein binding fragment is selected from a group of polypeptide sequences or a member of a group of polypeptide sequences consisting of:

a) one or two EDY sequences (SEQ ID NO:11 or SEQ ID NO 12) or homologs thereof wherein said homologs have at least 70, 80, 85, 90, 95, 97, 98 or 99% identity to SEQ ID NO:11 or SEQ ID NO 12,
b) the amino acids 252-264 (SEQ ID No:11) or 387-399 (SEQ ID No:12) of HDM2 or homologs thereof wherein said homologs have at least 70, 80, 85, 90, 95, 97, 98 or 99% identity to SEQ ID NO:11 or SEQ ID NO:12, or
c) the amino acids 257-259 or 392-394 of HDM2 (SEQ ID NO:5) or homologs thereof wherein said homologs have at least 70, 80, 85, 90, 95, 97, 98 or 99% sequence identity to SEQ ID No:5.

This invention further provides an isolated nucleic acid encoding said HDM2 fragments, wherein said fragments would minimally encode for the protein-binding domain as defined hereinbefore. The nucleic acid can be RNA or DNA, including cDNA and genomic DNA, in particular DNA.

In a particular embodiment said nucleic acid sequence is selected from a group of nucleotide sequences or a member of a group of nucleotide sequences comprising:

a) the N-terminal domain of HDM2 (SEQ ID NO:25),
b) nucleotide sequence 0-600 of HDM2 (SEQ ID NO:25),
c) the central domain of HDM2 (SEQ ID NO:25),
d) nucleotide sequence 600-1600 of HDM2 (SEQ ID NO:25),
e) nucleotide sequence 600-1100 of HDM2 (SEQ ID NO:25).
f) nucleotide sequence 1100-1600 of HDM2 (SEQ ID NO:25).

In a more particular embodiment said nucleic acid sequence is selected from a group of nucleotide sequences or a member of a group of nucleotide sequences comprising:

a) one or two EDY nucleotide sequences (SEQ ID NO:18 or SEQ ID NO 19) or homologs thereof, wherein said homologs have at least 70, 80, 85, 90, 95, 97, 98 or 99% identity to SEQ ID NO:18 or SEQ ID NO 19,
b) the nucleotide sequences 1050-1088 (SEQ ID No:18) or 1455-1493 (SEQ ID No:19) of HDM2 or homologs thereof wherein said homologs have at least 70, 80, 85, 90, 95, 97, 98 or 99% identity to SEQ ID NO:18 or SEQ ID NO:19, or
c) the nucleotide sequences 16-24 of SEQ ID NO:18 or SEQ ID NO 19 or homologs thereof wherein said homologs have at least 70, 80, 85, 90, 95, 97, 98 or 99% sequence identity to SEQ ID No:25.

In an even more particular embodiment said nucleic acid sequence is selected from a group of nucleotide sequences or a member of a group of nucleotide sequences consisting of:

a) one or two EDY nucleotide sequences (SEQ ID NO:18 or SEQ ID NO 19) or homologs thereof, wherein said homologs have at least 70, 80, 85, 90, 95, 97, 98 or 99% identity to SEQ ID NO:18 or SEQ ID NO 19,
b) the nucleotide sequences 1050-1088 (SEQ ID No:18) or 1455-1493 (SEQ ID No:19) of HDM2 or homologs thereof wherein said homologs have at least 70, 80, 85, 90, 95, 97, 98 or 99% identity to SEQ ID NO:18 or SEQ ID NO:19, or
c) the nucleotide sequences 16-24 of SEQ ID NO:18 or SEQ ID NO 19 or homologs thereof wherein said homologs have at least 70, 80, 85, 90, 95, 97, 98 or 99% sequence identity to SEQ ID No:25.

Given the identification and the characterization of the binding fragments within HDM2, it is also an object of the present invention to provide an isolated and purified nucleic acid molecule selected from a group or a member of a group consisting of:
(a) a nucleic acid molecule which is complementary to the above described polynucleotides;
(b) a nucleic acid molecule comprising at least 15 sequential bases of the above polynucleotides;
(c) a nucleic acid molecule that hybridizes under stringent conditions to the above described polynucleotide molecules; or
(d) a nucleic acid molecule encoding the protein binding fragment of HDM2 comprising a nucleotide sequence which is degenerated as a result of the genetic code to a nucleotide sequence of a polynucleotide of any of the above described polynucleotides.

The invention also provides a vector comprising the isolated nucleic acid molecules as defined above, as well as a host cell stably transformed with such a vector. Accordingly, in a specific embodiment said vector is an expression vector such as pGL3luc, pBLCAT5 (LMBP 2451), pGMCSFlacZ (LMBP 2979), pEGFP or pSEAPbasic (DMB 3115), wherein LMBP and DMB numbers refer to the accession numbers of these expression vectors at the Belgian Co-ordinated Collections of Micro-organisms. Included in the invention is also a host cell harboring a vector according to the invention. Such a host cell can be a prokaryotic cell, a unicellular eukaryotic cell or a cell derived from a multicellular organism. The host cell can thus e.g. be a bacterial cell, such as an E. coli cell; a yeast cell, such as Saccharomyces cerevisiae or Pichia pastoris, or a mammalian cell, such as HEK293 cells. The methods employed to effect introduction of the vector into the host cell are standard methods, well known to a person familiar with recombinant DNA methods.

Accordingly, in a further embodiment the present invention relates to the use of an isolated and purified polypeptide which encodes HDM2, a related protein or a protein binding fragment thereof, in an assay or a method of purification that makes use of the interaction of HDM2, related proteins or protein binding fragments thereof with another protein, the proteasome, proteasome subunits or protein binding fragments thereof.

In particular, the present invention encompasses the use in an assay or a method according to the invention, of the above described isolated and purified polypeptides.

In a further embodiment the present invention relates to the above described isolated and purified nucleic acid molecules which encode HDM2, related proteins or protein binding fragments thereof, wherein said nucleic acid molecule is either RNA, DNA, cDNA or genomic DNA, for use as a medicine.

Those skilled in the art will recognize that owing to the degeneracy of the genetic code, numerous “silent” substitutions of nucleotide base pairs could be introduced into the sequence identified as SEQ ID NO:18 or SEQ ID NO: 19 or the above identified fragments of said sequences, without altering the identity of the encoded amino acid(s) or protein products. All such substitutions are intended to be within the scope of the invention.

### Proteasome, Proteasome Subunit and, Protein Binding Fragments Thereof

This invention provides for isolated polypeptide molecules encoding protein binding regions of a proteasome or proteasome subunit, said fragments comprising at least 10 amino acids that are contiguous in the parent protein, but may desirably contain at least 11, 12, 13, 14, 15, 20, 30, 40, 60, 80, 100, 150, 200, 250, 300, or 350 amino acids that are contiguous in the parent protein and wherein said fragments are capable of binding proteins such as f.e. HDM2, or a proteasome binding fragment thereof.

In a particular embodiment said protein binding fragment of a proteasome or proteasome subunit is selected from a group of polypeptide sequences or a member of a group of polypeptide sequences comprising but not limited to the protein binding fragments present in the proteasome subunits S6A, S6B, S5A, S2 or S4 or homologs thereof wherein said homologs have at least 70, 80, 85, 90, 95, 97, 98 or 99% identity to SEQ ID 1, SEQ ID 2, SEQ ID 3, SEQ ID 4 or SEQ ID 6.

In a more particular embodiment said protein binding fragment is selected from a group of polypeptide sequences or a member of a group of polypeptide sequences comprising:

a) amino acid sequences SEQ ID No:7, SEQ ID No:8, SEQ ID No:9, SEQ ID No:10 or SEQ ID No:13 or homologs thereof wherein said homologs have at least 70, 80, 85, 90, 95, 97, 98 or 99% identity to any one of SEQ ID No:7, SEQ ID No:8, SEQ ID No:9, SEQ ID No:10 or SEQ ID No:13,
b) amino acids 413-425 (SEQ ID NO:7) of S6A, amino acids 356-368 (SEQ ID NO:8) of S6B, amino acids 318-331 (SEQ ID NO:9) of S5A, amino acids 432-444 (SEQ ID NO:10) of S2 or amino acids 431-440 (SEQ ID NO:13) of S4 or homologs thereof wherein said homologs have at least 70, 80, 85, 90, 95, 97, 98 or 99% sequence identity to SEQ ID NO:7, SEQ ID NO:8, SEQ ID NO:9, SEQ ID NO:10 or SEQ ID No:13, or
c) the amino acids 418-420 of S6A (SEQ ID NO:1), the amino acids 418-420 of S6A (SEQ ID NO:2), the amino acids 361-363 of S6B (SEQ ID NO:3), the amino acids 323-326 of S5A (SEQ ID NO:4), the amino acids 437-439 of S2 (SEQ ID NO:6) or the amino acids 436-439 of S4 (SEQ ID NO:13) or homologs thereof wherein said homologs have at least 70, 80, 85, 90, 95, 97, 98 or 99% sequence identity to SEQ ID NO:1, SEQ ID NO:2, SEQ ID NO:3, SEQ ID NO:4 or SEQ ID No:6.

In an even more particular embodiment said protein binding fragment is selected from a group of polypeptide sequences or a member of a group of polypeptide sequences consisting of:

a) amino acid sequences SEQ ID No:7, SEQ ID No:8, SEQ ID No:9, SEQ ID No:10 or SEQ ID No:13 or homologs thereof wherein said homologs have at least 70, 80, 85, 90, 95, 97, 98 or 99% identity to any one of SEQ ID No:7, SEQ ID No:8, SEQ ID No:9, SEQ ID No:10 or SEQ ID No:13,
b) amino acids 413-425 (SEQ ID NO:7) of S6A, amino acids 356-368 (SEQ ID NO:8) of S6B, amino acids 318-331 (SEQ ID NO:9) of S5A, amino acids 432-444 (SEQ ID NO:10) of S2 or amino acids 431-440 (SEQ ID NO:13) of S4 or homologs thereof wherein said homologs have at least 70, 80, 85, 90, 95, 97, 98 or 99% sequence identity to SEQ ID NO:7, SEQ ID NO:8, SEQ ID NO:9, SEQ ID NO:10 or SEQ ID No:13, or
c) the amino acids 418-420 of S6A (SEQ ID NO:1), the amino acids 418-420 of S6A (SEQ ID NO:2), the amino acids 361-363 of S6B (SEQ ID NO:3), the amino acids 323-326 of S5A (SEQ ID NO:4), the amino acids 437-439 of S2 (SEQ ID NO:6) or the amino acids 436-439 of S4 (SEQ ID NO:13) or homologs thereof wherein said homologs have at least 70, 80, 85, 90, 95, 97, 98 or 99% sequence identity to SEQ ID NO:1, SEQ ID NO:2, SEQ ID NO:3, SEQ ID NO:4 or SEQ ID No:6.

This invention further provides an isolated nucleic acid encoding the protein-binding domains as defined hereinbefore. The nucleic acid can be RNA or DNA, including cDNA and genomic DNA, in particular DNA and are in a further embodiment selected from the nucleic acid sequences encoding protein binding domains and HDM2 binding domains from proteasome subunits.

In a particular embodiment, the isolated nucleic acid molecules encode the binding fragments present in the proteasome subunits S6a, S6b, S5a, S4 or S2 consisting of the amino acid sequences selected from the group consisting of SEQ ID No:1, SEQ ID No:2, SEQ ID No:3, SEQ ID No:4 or SEQ ID No:6. In a more particular embodiment said isolated nucleic acid encode fragments comprising the protein binding region consisting of the amino acids 413-425 (SEQ ID NO:7) of S6A, amino acids 356-368 (SEQ ID NO:8) of S6B, amino acids 318-331 (SEQ ID NO:9) of S5A, amino acids 432-444 (SEQ ID NO:10) of S2 or amino acids 431-440 (SEQ ID NO:13) of S4 or homologs thereof wherein said homologs have at least 70, 80, 85, 90, 95, 97, 98 or 99% sequence identity to SEQ ID NO:7, SEQ ID NO:8, SEQ ID NO:9, SEQ ID NO:10 or SEQ ID No:13 or a nucleic acid sequence having at least 70, 80, 90, 95, 97 or 98% sequence identity to any of the aforementioned nucleic acid sequences.

In a more particular embodiment said nucleic acid sequence is selected from a group of nucleotide sequences or a member of a group of nucleotide sequences comprising:

a) the EDY nucleotide sequences of proteasome subunits S6a, S6b, S5a, S4 or S2 wherein said homologs have at least 70, 80, 85, 90, 95, 97, 98 or 99% identity to SEQ ID NO:14, SEQ ID NO:15, SEQ ID NO:16, SEQ ID NO:17 or SEQ ID NO 20.
b) the nucleotides sequence 1431-1469 of SEQ ID No:21, 1103-1141 of SEQ ID No:22, 1014-1055 of SEQ ID No:23, 1327-1365 of SEQ ID No:24 or 1339-1371 of SEQ ID No:26 of proteasome subunits S6a, S6b, S5a, S4 or S2 or homologs thereof wherein said homologs have at least 70, 80, 85, 90, 95, 97, 98 or 99% identity to SEQ ID NO:21, ID NO:22, ID NO:23, ID NO:24 or SEQ ID NO:16.
c) the nucleotides sequence 1-39 of SEQ ID NO:14, SEQ ID NO:15 or SEQ ID NO:17, the nucleotides sequence 1-42 of SEQ ID NO:16 or the nucleotides sequence 1-33 of SEQ ID NO:20 or homologs thereof wherein said homologs have at least 70, 80, 85, 90, 95, 97, 98 or 99% sequence identity to SEQ ID NO:14, SEQ ID NO:15, SEQ ID NO:16, SEQ ID NO:17 or SEQ ID NO 20.
d) the nucleotides sequence 16-24 of SEQ ID NO:14, SEQ ID NO:15 or SEQ ID NO:17, the nucleotides sequence 16-27 of SEQ ID NO:16 or SEQ ID NO:20 or homologs thereof wherein said homologs have at least 70, 80, 85, 90, 95, 97, 98 or 99% sequence identity to SEQ ID NO:14, SEQ ID NO:15, SEQ ID NO:16, SEQ ID NO:17 or SEQ ID NO 20.

In an even more particular embodiment said nucleic acid sequence is selected from a group of nucleotide sequences or a member of a group of nucleotide sequences consisting of:

a) EDY nucleotide sequences of proteasome subunits S6a, S6b, S5a, S4 or S2 wherein said homologs have at least 70, 80, 85, 90, 95, 97, 98 or 99% identity to SEQ ID NO:14, SEQ ID NO:15, SEQ ID NO:16, SEQ ID NO:17 or SEQ ID NO 20.
b) the nucleotides sequence 1431-1469 of SEQ ID No:21, 1103-1141 of SEQ ID No:22, 1014-1055 of SEQ ID No:23, 1327-1365 of SEQ ID No:24 or 1339-1371 of SEQ ID No:26 of proteasome subunits S6a, S6b, S5a, S4 or S2 or homologs thereof wherein said homologs have at least 70, 80, 85, 90, 95, 97, 98 or 99% identity to SEQ ID NO:21, ID NO:22, ID NO:23, ID NO:24 or SEQ ID NO:16.
c) the nucleotides sequence 1-39 of SEQ ID NO:14, SEQ ID NO:15 or SEQ ID NO:17, the nucleotides sequence 1-42 of SEQ ID NO:16 or the nucleotides sequence 1-33 of SEQ ID NO:20 or homologs thereof wherein said homologs have at least 70, 80, 85, 90, 95, 97, 98 or 99% sequence identity to SEQ ID NO:14, SEQ ID NO:15, SEQ ID NO:16, SEQ ID NO:17 or SEQ ID NO 20.
d) the nucleotides sequence 16-24 of SEQ ID NO:14, SEQ ID NO:15 or SEQ ID NO:17, the nucleotides sequence 16-27 of SEQ ID NO:16 or SEQ ID NO:20 or homologs thereof wherein said homologs have at least 70, 80, 85, 90, 95, 97, 98 or 99% sequence identity to SEQ ID NO:14, SEQ ID NO:15, SEQ ID NO:16, SEQ ID NO:17 or SEQ ID NO 20.

Given the identification and the characterization of the proteasome consensus-binding region, it is also an object of the present invention to provide an isolated and purified nucleic acid molecule selected from a group consisting of:
a) a nucleic acid molecule which is complementary to the above polynucleotides;
b) a nucleic acid molecule comprising at least 15 sequential bases of the above polynucleotide;
c) a nucleic acid molecule that hybridizes under stringent conditions to the above polynucleotide molecules; or
d) a nucleic acid molecule encoding the protein binding region of proteasome subunits and their counterparts comprising a nucleotide sequence which is degenerated as a result of the genetic code to a nucleotide sequence of the above polynucleotides.

The invention also provides a vector comprising the isolated nucleic acid molecules as defined above, as well as a host cell stably transformed with such a vector. Accordingly, in a specific embodiment said vector is an expression vector such as pGL3luc, pBLCAT5 (LMBP 2451), pGMCSFlacZ (LMBP 2979), pEGFP or pSEAPbasic (DMB 3115), wherein LMBP and DMB numbers refer to the accession numbers of these expression vectors at the Belgian Co-ordinated Collections of Micro-organisms. Included in the invention is also a host cell harboring a vector according to the invention. Such a host cell can be a prokaryotic cell, a unicellular eukaryotic cell or a cell derived from a multicellular organism. The host cell can thus e.g. be a bacterial cell, such as an E. coli cell; a yeast cell, such as Saccharomyces cerevisiae or Pichia pastoris, or a mammalian cell, such as HEK293 cells. The methods employed to effect introduction of the vector into the host cell are standard methods, well known to a person familiar with recombinant DNA methods.

Accordingly, in a further embodiment the present invention relates to the use of an isolated and purified polypeptide which encodes the proteasome, a proteasome subunit or a protein binding fragment thereof, in an assay that makes use of the interaction of HDM2 and the related peptides with the proteasome, proteasome subunits or protein binding fragments thereof.

In particular, the present invention encompasses the use in an assay according to the invention of the above described isolated and purified polypeptide encoding a proteasome, proteasome subunit or a protein-binding fragment thereof.

It is also an embodiment of the present invention to provide the above described isolated and purified polypeptide and nucleic acid molecules encoding proteasome subunits, protein-binding fragments or a HDM2 binding fragments thereof for use as a medicine.

Those skilled in the art will recognize that owing to the degeneracy of the genetic code, numerous “silent” substitutions of nucleotide base pairs could be introduced into the sequence identified as SEQ ID NO:14, SEQ ID NO:15, SEQ ID NO:16, SEQ ID NO:17 or SEQ ID NO 20 or the above identified fragments of said sequences, without altering the identity of the encoded amino acid(s) or protein products. All such substitutions are intended to be within the scope of the invention.

The percentage identity of nucleic acid and polypeptide sequences can be calculated using commercially available algorithms which compare a reference sequence with a query sequence. The following programs (provided by the National Center for Biotechnology Information) may be used to determine homologies/identities: BLAST, gapped BLAST, BLASTN and PSI-BLAST, which may be used with default parameters.

The algorithm GAP (Genetics Computer Group, Madison, Wis.) uses the Needleman and Wunsch algorithm to align two complete sequences that maximizes the number of matches and minimizes the number of gaps. Generally, the default parameters are used, with a gap creation penalty=12 and gap extension penalty=4.

Another method for determining the best overall match between a nucleic acid sequence or a portion thereof, and a query sequence is the use of the FASTDB computer program based on the algorithm of Brutlag et al (Comp. App. Biosci., 6; 237-245 (1990)). The program provides a global sequence alignment. The result of said global sequence alignment is in percent identity. Suitable parameters used in a FASTDB search of a DNA sequence to calculate percent identity are: Matrix=Unitary, k-tuple=4, Mismatch penalty=1, Joining Penalty=30, Randomization Group Length=0, Cutoff Score=1, Gap Penalty=5, Gap Size Penalty=0.05, and Window Size=500 or query sequence length in nucleotide bases, whichever is shorter. Suitable parameters to calculate percent identity and similarity of an amino acid alignment are: Matrix=PAM 150, k-tuple=2, Mismatch Penalty=1, Joining Penalty=20, Randomization Group Length=0, Cutoff Score=1, Gap Penalty=5, Gap Size Penalty=0.05, and Window Size=500 or query sequence length in nucleotide bases, whichever is shorter.

The proteins and the peptides according to the invention includes all possible conservative amino acid changes, wherein “conservative amino acid changes” refers to a replacement of one or more amino acid residue(s) in a parent receptor protein or peptide without affecting the biological activity of the parent molecule based on the art recognized substitutability of certain amino acids (See e.g. M. Dayhoff, In Atlas of Protein Sequence and Structure, Vol. 5, Supp. 3, pgs 345-352, 1978).

Those skilled in the art will recognize that the polypeptides according to the invention, i.e. the HDM2 proteins, the related proteins, the protein binding fragments thereof, the proteasome, proteasome subunits and the protein binding fragments thereof, could be obtained by a plurality of recombinant DNA techniques including, for example, hybridization, polymerase chain reaction (PCR) amplification, or de novo DNA synthesis (See e.g., T. Maniatis et al. Molecular Cloning: A Laboratory Manual, 2d Ed. Chap. 14 (1989)).

The peptides and derivatives of the present invention can be readily prepared according to well-established, standard liquid or, preferably, solid-phase peptide synthesis methods, general descriptions of which are broadly available, or they may be prepared in solution, by the liquid phase method or by any combination of solid-phase, liquid phase and solution chemistry.

A polypeptide according to the present invention may be isolated and/or purified (e.g. using an antibody) for instance after production by expression from encoding nucleic acid.

The isolated and/or purified polypeptide and polynucleotides may be used in formulation of a composition, which may include at least one additional component, for example a pharmaceutical composition including a pharmaceutically acceptable excipient, vehicle or carrier.

A polypeptide according to the present invention may be used as an immunogen or otherwise in obtaining specific antibodies. Antibodies are useful in purification and other manipulation of polypeptides, diagnostic screening and therapeutic contexts. Antibodies to the polypeptides of the present invention may, advantageously, be prepared by techniques which are known in the art. For example, polyclonal antibodies may be prepared by inoculating a host animal such as a mouse with the growth factor or an epitope thereof and recovering immune serum. Monoclonal antibodies may be prepared according to known techniques such as described by Kohler R. and Milstein C., Nature (1975) 256, 495-497.

### Assays

Assays of the present invention can be designed in many formats generally known in the art of screening compounds for biological activity or for binding proteins.

Polypeptides and polynucleotides of the present invention are responsible for one or more biological functions, including one or more disease states, in particular the diseases hereinbefore mentioned. It is therefore desirable to devise screening methods to identify compounds which interfere e,g, with the UPS-pathway.

The assays of the present invention advantageously exploit the fact that disturbance of the interaction between HDM2 and the proteasome, proteasome subunits and the binding fragments thereof affect e.g. the downstream-targeted degradation of ubiquitinilated proteins.

Furthermore, the present invention includes methods of identifying compounds that specifically bind to HDM2, related proteins or protein binding fragments thereof, wherein said compounds affect the interaction between HDM2, related proteins or protein binding fragments thereof and another protein, the proteasome or its subunits.

The binding of a compound to HDM2, related proteins or a protein binding fragment thereof can only require a simple linear stretch of amino acids, can comprise for example a modified (e.g. phosphorylation or hydroxylation) or conformationally sensitive motif or can require several amino acid sequences distributed in a specific way along the protein.

A first method of the present invention differ from those described in the art because the assay incorporates at least one step wherein the interaction of HDM2, a related protein or the protein binding fragments thereof with a compound, protects a Hdm2 binding protein against proteolysis. More specifically the compound protects a Hdm2 binding protein against proteolysis by the UPS-pathway.

Thus, the present invention provides for a method to identify compounds that affect binding of Hdm2, a related protein or a protein binding fragment thereof to the proteasome, a proteasome subunit or a protein binding fragment thereof, said method comprising:
a) contacting the compound to be tested with a HDM2 protein or a related protein or a protein binding fragment thereof and
b) determining whether said compound affects the proteolysis of a Hdm2 binding protein by the ubiquitin-proteasome proteolysis pathway.

In an embodiment of the invention, proteolysis of a HDM2 binding protein can be effected in cells, cell lysates, in vitro UPS systems or in vivo systems.

In another embodiment the Hdm2 binding protein is p53.

In another embodiment the Hdm2 binding protein is other than p53

A second method of the invention may simply measure the binding of a candidate compound to the polypeptide, or to cells or membranes bearing the polypeptide, or a fusion protein thereof by means of a label directly or indirectly associated with the polypeptide. Those assays differ from the art in that they apply the ED(X)Y sequences or their counterparts.

Therefore, in an embodiment, the screening method comprises labelled HDM2, labelled related proteins or labelled fragments thereof, wherein said label is used to measure the effect of the test compound on the amount of HDM2, related proteins or protein binding fragments thereof bound to the proteasome subunit or protein binding fragments thereof.

Accordingly, the present invention provides for a method to identify compounds that affect binding of Hdm2, a related protein or a protein binding fragment thereof to the proteasome, a proteasome subunit or a protein binding fragment thereof, said method comprising:
a) incubating the proteasome subunit or protein binding fragments thereof with labelled HDM2, labelled related proteins or labelled protein binding fragments thereof,
b) adding the test compound to the incubation mixture, and
c) measuring the effect of the test compound on the amount of labelled HDM2, labelled related proteins or labelled protein binding fragments thereof bound to the proteasome subunit or protein binding fragments thereof.

Alternatively, the screening method comprises labelled proteasome subunit or labelled protein binding fragments thereof wherein said label is used to measure the effect of the test compound on the amount of HDM2, related proteins or protein binding fragments thereof bound to the proteasome subunit or protein binding fragments thereof.

Accordingly, the present invention provides for a method to identify compounds that affect binding of Hdm2, a related protein or a protein binding fragment thereof to the proteasome, a proteasome subunit or a protein binding fragment thereof, said method comprising:
a) incubating the labelled proteasome subunit or labelled protein binding fragments thereof with HDM2, related proteins or protein binding fragments thereof,
b) adding the test compound to the incubation mixture, and
c) measuring the effect of the test compound on the amount of labelled proteasome subunit or labelled protein binding fragments thereof bound to HDM2, related proteins or protein binding fragments thereof.

Examples of possible binding assays are the immunoprecipitation assay as provided in the examples hereinafter or the use of a surface plasmon resonance effect exploited by the Biacore instrument (Malmqvist M., Biochem Soc Trans. 1999 February; 27(2):335-40). In the latter FLAG-tagged or His-tagged version of the polypeptides of this invention could be attached to the biosensor chip of a Biacore and binding of binding partner examined in the presence and absence of compounds to identify competitors of the binding site. For example, in one embodiment the proteasome subunit as defined hereinbefore would be immobilized on the Biacore chip using a Flag tag and the binding of a protein or fragments thereof would be examined in the presence and absence of compounds to identify competitors of the binding site. Alternatively, the HDM2 binding fragment of proteasomes or homologs thereof as defined hereinbefore would be immobilized on the Biacore chip using a Flag tag and the binding of HDM2 or the proteasome binding fragment thereof would be examined in the presence and absence of compounds to identify competitors of the binding site.

Tagging of the polypeptides according to the invention, is also useful to immobilize said molecules in conventional filter-binding assays (eg. Using Brandel filter assay equipment) or in high throughput Scintillation Proximity type binding assays (SPA and Cytostar-T flashplate technology; Amersham Pharmacia Biotech) to detect binding of radio-labelled ligand and displacement of such radio-ligands by competitors for the binding site. Radioactivity can be measured with Packard Topcount, or similar instrumentation, capable of making rapid measurements from 96-, 384-, 1536-microtitre well formats. SPA/Cytostar-T technology is particularly amenable to high throughput screening and therefore this technology is suitable to use as a screen for compounds able to displace standard ligands.

Further, these screening methods may test whether the candidate compound results in a signal generated by activation or inhibition of an enzyme, using detection systems appropriate to enzymatic activity of said enzyme. Enzymatic activity is generally assessed using an appropriate substrate that upon processing provides a measurable signal.

Compounds may be identified from a variety of sources, for example, cells, cell-free preparations, chemical libraries, and natural product mixtures. Such agonists or antagonists so-identified may be natural or modified peptides, ligands, enzymes, etc., as the case may be, of the receptor polypeptide; or may be structural or functional mimetics thereof (see Coligan et al., Current Protocols in Immunology 1 (2):Chapter 5 (1991)).

In an embodiment of the invention the compound that affect binding of HDM2, a related protein or a protein binding fragment thereof to the proteasome, a proteasome subunit or a protein binding fragment thereof is a peptide, more particular one of the polypeptides described hereinabove or a fusion protein of said peptide.

In a further aspect the present invention provides a method for isolating proteins e.g. HDM2-binding proteins or related proteins, proteasome subunits or proteasome-binding proteins from a cellular fraction containing the same, comprising contacting the cellular fraction with the peptides or peptide mimetics of the invention immobilized to a solute substrate and eluting the binding partner therefrom.

In another embodiment of the invention the compound that affect binding of HDM2, a related protein or a protein binding fragment thereof to the proteasome, a proteasome subunit or a protein binding fragment thereof, is a small molecule, more particular compound JNJ #1

It will be readily appreciated by the skilled artisan that the discovery of the ED(X)Y sequences may also be used in a method for the structure-based or rational design of an antagonist of the binding between HDM2 or related proteins to a proteasome subunit, by:
a) probing the proteasome subunit or protein binding fragments thereof with HDM2, related proteins or protein binding fragments thereof,
b) identifying contacting atoms in the binding site of the proteasome subunit or protein binding fragments thereof that interact with HDM2, related proteins or protein binding fragments thereof or vice versa,
c) design test compounds that interact with the atoms identified in (b), and
d) contact said designed test compound with a proteasome subunit, a protein binding fragment thereof, HDM2, related proteins or protein binding fragments thereof to measure the capability of said compound to modulate the interaction between HDM2 and the proteasome or to modulate ubiquitinin-proteasome proteolysis.

Molecular modeling techniques are known in the art, including both hardware and software appropriate for creating and utilizing models of receptors and enzyme conformations.

Numerous computer programs are available and suitable for the processes of computer modeling, model building and computationally identifying, selecting and evaluating potential interacting compounds in the methods described herein. These include for example, GRID (available from Oxford University, UK), MCSS (available from Accelrys, Inc., San Diego, Calif.), AUTODOCK (available from Oxford Molecular Group), FLEX X (available form Tripos, St. Louis, Mo.), DOCK (available from University of California, San Francisco, Calif.), CAVEAT (available from University of California, Berkeley), HOOK (available from Accelrys, Inc., San Diego, Calif.) and 3D database systems such as MACCS-3D (available from MDL Information Systems, San Leandro, Calif.), UNITY (available from Tripos, St. Louis, Mo.) and CATALYST (available from Accelrys, Inc., San Diego, Calif.). Potential candidate substances may also be computationally designed “de novo’ using software packages as LUDI (available from Biosym Technologies, San Diego, Calif.), LEGEND (available from Accelrys, Inc, San Diego, Calif.) and LEAPFROG (available from Tripos, St. Louis, Mo.). Compound deformation energy and electrostatic repulsion, may be analysed using programs such as GAUSSIAN 92, AMBER, QUANTA/CHARMM and INSIGHT II/DISCOVER. These computer evaluation and modeling techniques may be performed on any suitable hardware including for example, workstations available from Silicon Graphics, Sun Microsystems and others. These modeling techniques, methods, hardware and software packages are representative and are not intended to be a comprehensive listing. Other modeling techniques known in the art may also be employed in accordance with this invention. See for example, N. C. Cohen, Molecular Modeling in Drug Design, Academic Press (1996).

In one embodiment of the present invention, the three-dimensional structure of the proteasome binding domain is generated using the atomic coordinates of the S5a proteasome subunit (Protein Database 1EUL)+/−a root mean square deviation of the backbone atoms of said amino acids of not more that 10 Å, preferably not more that 5 Å.

In this screening, the quality of fit of such compounds to the binding site may be judged either by shape complementarity or by estimated interaction energy (Meng, E. C. et al., J. Coma. Chem 13:505-524 (1992)).

Other molecular modeling techniques may also be employed in accordance with this invention. See, e.g., Cohen, N. C. et al., “Molecular Modeling Software and Methods for Medicinal Chemistry, J. Med. Chem. 33:883-894 (1990). See also, Navia, M. A. and M. A. Murcko, “The Use of Structural Information in Drug Design,” Current Opinions in Structural Biology, 2, pp. 202-210 (1992).

Once a compound has been designed or selected by the above methods, the affinity with which that compound may bind or associate with a proteasome binding domain or a protein binding domain may be tested and optimized by computational evaluation and/or by testing biological activity after synthesizing the compound. Inhibitors or compounds may interact with the proteasome binding domain or the protein binding domain in more than one conformation that is similar in overall binding energy. In those cases, the deformation energy of binding is taken to be the difference between the energy of the free compound and the average energy of the conformations observed when the compound binds to a proteasome binding domain or a protein binding domain.

A compound designed or selected as binding or associating with a proteasome binding domain or a protein binding domain may be further computationally optimized so that in its bound state it would preferably lack repulsive electrostatic interaction with the binding domains. Such non-complementary (e.g., electrostatic) interactions include repulsive charge-charge, dipole-dipole and charge-dipole interactions. Specifically, the sum of all electrostatic interactions between the inhibitor and a proteasome binding domain or a protein binding domain when the inhibitor is bound, preferably make a neutral or favorable contribution to the enthalpy of binding. Weak binding compounds will also be designed by these methods so as to determine SAR. See, for example, U.S. Appl. Nos. 60/275,629; 60/331,235; 60/379,617; and, 10/097,249.

Specific computer software is available in the art to evaluate compound deformation energy and electrostatic interaction. Examples of programs designed for such uses include: Gaussian 92, revision C (M. J. Frisch, Gaussian, Inc., Pittsburgh, Pa., COPYRGT 1992); AMBER, version 4.0 (P. A. Kollman, University of California at San Francisco, COPYRGT 1994); QUANTA/CHARMM (Molecular Simulations, Inc., Burlington, Mass. COPYRGT 1994); and Insight II/Discover (Biosysm Technologies Inc., San Diego, Calif. COPYRGT 1994). Other hardware systems and software packages will be known to those skilled in the art.

Once a compound that associates with a proteasome binding domain or a protein binding domain has been optimally selected or designed, as described above, substitutions may then be made in some of its atoms or side groups in order to improve or modify its binding properties. Generally, initial substitutions are conservative, i.e., the replacement group will have approximately the same size, shape, hydrophobicity and charge as the original group. It should, of course, be understood that components known in the art to alter conformation may be avoided. Such substituted chemical compounds may then be analyzed for efficiency of fit to a proteasome binding domain or a protein binding domain by the same computer methods described in detail, above.

The present invention also provides peptidomimetics of the polypeptides described herein. Peptide analogs are commonly used in the pharmaceutical industry as non-peptide drugs with properties analogous to those of the template peptide. These types of non-peptide compound are termed “peptide mimetics” or “peptidomimetics” (Fauchere (1986) Adv. Drug Res. 15: 29; Veber and Freidinger (1985) TINS p. 392; and Evans et al. (1987) J. Med. Chem. 30: 1229) and are usually developed with the aid of computerized molecular modeling. Peptide mimetics that are structurally similar to therapeutically useful peptides may be used to produce an equivalent therapeutic or prophylactic effect.

The present invention further provides systems, particularly computer-based systems, which contain the sequence and/or structure coordinates described herein. Such systems are designed to do structure determination and rational drug design for a proteasome binding domain or a protein binding domain. The computer-based systems refer to the hardware means, software means and data storage means used to analyze the sequence and/or structure coordinates of the present invention in any of the computer methods described in detail, above. The minimum hardware means of the computer-based system of the present invention comprises a central processing unit (CPU), input means, output means and data storage means. A skilled person can readily appreciate which of the currently available computer-based systems are suitable for use in the present invention.

It is accordingly an object of the present invention to provide computer readable data storage medium containing the structure coordinates described herein. As used herein, “computer readable data storage medium” refers to any medium which can be read or accessed directly by a computer. Such media include, but are not limited to: magnetic storage media, such as floppy disks, hard disc storage media and magnetic tape; optical storage media such as optical discs or CD-ROM; electrical storage media such as RAM and ROM; and hybrids of these categories such as magnetical/optical storage media.

### Therapeutic Use

In general the polypeptides (the ED(X)Y sequences and their counterparts) and polynucleotides of the invention may be employed for therapeutic and prophylactic purposes for such diseases as hereinbefore and hereinafter mentioned.

Therefore, the present invention relates to the use of one or more members of the group selected from the polypeptides, the peptides, the peptidomimetics, the proteasome subunits, protein binding fragments thereof, HDM2, related proteins, protein binding fragments thereof and the polynucleotides as a medicine and for use in the treatment of inhibiting the growth of tumours.

Thus the present invention relates to the use of the above described polypeptides and polynucleotides for the manufacture of a medicament for the treatment of cancer and leukemia.

The present invention further relates to the use of said polypeptides and said polynucleotides as a medicine and for use in the treatment of inhibiting the growth of tumours.

Examples of tumours which may be inhibited, but are not limited to, lung cancer (e.g. adenocarcinoma and including non-small cell lung cancer), pancreatic cancers (e.g. pancreatic carcinoma such as, for example exocrine pancreatic carcinoma), colon cancers (e.g. colorectal carcinomas, such as, for example, colon adenocarcinoma and colon adenoma), prostate cancer including the advanced disease, hematopoietic tumours of lymphoid lineage (e.g. acute lymphocytic leukemia, B-cell lymphoma, Burkitt's lymphoma), myeloid leukemias (for example, acute myelogenous leukemia (AML)), thyroid follicular cancer, myelodysplastic syndrome (MDS), tumours of mesenchymal origin (e.g. fibrosarcomas and rhabdomyosarcomas), melanomas, teratocarcinomas, neuroblastomas, gliomas, benign tumour of the skin (e.g. keratoacanthomas), breast carcinoma (e.g. advanced breast cancer), kidney carcinoma, ovary carcinoma, bladder carcinoma and epidermal carcinoma.

Thus, in a further aspect, the present invention provides a method for preventing, treating or ameliorating a medical condition related to the UPS activity which comprises administering to a mammalian subject a therapeutically effective amount of a UPS modulating compound as described above, including but not limited to the ED(X)Y sequences, their protein counterparts and the polynucleotides encoding for said polypeptides, optionally in combination with a pharmaceutically acceptable carrier, in an amount effective to modulate the UPS activity. Such carriers include, but are not limited to, saline, buffered saline, dextrose, water, glycerol, ethanol, and combinations thereof. The invention further relates to pharmaceutical packs and kits comprising one or more containers filled with one or more of the ingredients of the aforementioned compositions of the invention. Polypeptides and polynucleotides of the present invention may be employed alone or in conjunction with other compounds, such as therapeutic compounds.

The compound or composition will be adapted to the route of administration, for instance by a systemic or an oral route. Preferred forms of systemic administration include injection, typically by intravenous injection. Other injection routes, such as subcutaneous, intramuscular, or intraperitoneal, can be used. Alternative means for systemic administration include transmucosal and transdermal administration using penetrants such as bile salts or fusidic acids or other detergents. In addition, if a polypeptide or other compounds of the present invention can be formulated in an enteric or an encapsulated formulation, oral administration may also be possible. Administration of these compounds may also be topical and/or localized, in the form of patches, salves, pastes, gels, and the like.

The compound, composition or formulation to be administered will, in any event, contain a quantity of the active compound(s) in an amount effective to alleviate the symptoms of the subject being treated.

The exact dosage and frequency of administration of the present compounds depends on the particular compound used, the particular condition being treated, the severity of the condition being treated, the age, weight, gender, diet, time of administration and general physical condition of the particular patient, the mode of administration as well as other medication the individual may be taking, as is well known to those skilled in the art. Furthermore, it is evident that the effective daily amount may be lowered or increased depending on the response of the treated subject and/or depending on the evaluation of the physician prescribing the compounds of the instant invention.

Dosage forms or compositions containing active ingredient in the range of 0.25 to 95% with the balance made up from non-toxic carrier may be prepared. Depending on the mode of administration, the pharmaceutical composition will preferably comprise from 0.05 to 99% by weight, more preferably from 0.1 to 70% by weight of the active ingredients, and, from 1 to 99.95% by weight, more preferably from 30 to 99.9 weight % of a pharmaceutically acceptable carrier, all percentages being based on the total composition.

For oral administration, a pharmaceutically acceptable non-toxic composition is formed by the incorporation of any of the normally employed excipients, such as, for example, pharmaceutical grades of mannitol, lactose, cellulose, cellulose derivatives, sodium crosscarmellose, starch, magnesium stearate, sodium saccharin, talcum, glucose, sucrose, magnesium, carbonate, and the like. Such compositions take the form of solutions, suspensions, tablets, pills, capsules, powders, sustained release formulations and the like. Such compositions may contain 1%-95% active ingredient, more preferably 2-50%, most preferably 5-8%.

Parenteral administration is generally characterized by injection, either subcutaneously, intramuscularly or intravenously. Injectables can be prepared in conventional forms, either as liquid solutions or suspensions, solid forms suitable for solution or suspension in liquid prior to injection, or as emulsions. Suitable excipients are, for example, water, saline, dextrose, glycerol, ethanol or the like. In addition, if desired, the pharmaceutical compositions to be administered may also contain minor amounts of non-toxic auxiliary substances such as wetting or emulsifying agents, pH buffering agents and the like, such as for example, sodium acetate, sorbitan monolaurate, triethanolamine oleate, triethanolamine sodium acetate, etc.

The percentage of active compound contained in such parental compositions is highly dependent on the specific nature thereof, as well as the activity of the compound and the needs of the subject. However, percentages of active ingredient of 0.1% to 10% in solution are employable, and will be higher if the composition is a solid which will be subsequently diluted to the above percentages. Preferably, the composition will comprise 0.2-2% of the active agent in solution.

Finally, this invention provides for an article of manufacture comprising a packaging and a pharmaceutical agent, wherein (a) the pharmaceutical agent is identified using an assay of the present invention, and (b) the packaging comprises a label indicating the use of the agent for treating a cell proliferative disorder in a subject. In particular as an anti-cancer medicine.

This invention will be better understood by reference to the Experimental Details that follow, but those skilled in the art will readily appreciate that these are only illustrative of the invention as described more fully in the claims that follow thereafter. Additionally, throughout this application, various publications are cited. The disclosure of these publications is hereby incorporated by reference into this application to describe more fully the state of the art to which this invention pertains.

### EXPERIMENTAL

### Materials and Methods

### Plasmids

The plasmids pDWM659 encoding Myc-MDM2 as well as the plasmids pcDNA3-Mdm2 and pcDNA3-p53 have been described previously (Blattner et al., 2002). Full length GST-HDM2, GST-HDM2 fragments GST-HDM2-1-206, GST-HDM2-293-493, GST-S6b-EDY peptide and GST-HDM2-EDY peptide were created by amplifying the respective sequences by PCR using primers containing appropriate restriction sites. GST-HDM2-100-200 was ordered with Abnova Corporation—Catalog number H00004193-Q01. The PCR fragments were digested with EcoRI and NotI and cloned into the pGex-4T-2 vector. GST-HdmX was created by amplifying HdmX by PCR using reversely transcribed RNA as a template and primers encoding appropriate restriction sites. The PCR fragment was ligated into the pGEX-4T-2 vector. Expression and purification of proteins were performed according to the recommendation of the supplier of the pGEX-4T-2 vector (Amersham). Baculoviruses expressing E1 and the plasmid for UbCH5 were kindly provided by Martin Scheffner, Konstanz. Baculoviruses encoding Flag-Mdm2 and Flag-p53 have been described previously (Brignone et al., 2004).

### Antibodies

The following antibodies were used: the anti-myc antibody 9E10 (Santa Cruz), the anti-proliferating nuclear cell antigen (PCNA) monoclonal antibody PC10 (Santa Cruz), the anti-S8 antibody clone P45-110 (Biomol), an anti-S6b rabbit polyclonal antibody (Biomol), the anti-HDM2 antibodies C18, SMP14 (Santa Cruz) and 4B2 (Oncogene Sciences), the anti-p53 antibody DO-1 (Santa Cruz), and the HRP-coupled anti-mouse (P0161) and anti-rabbit (P0448) antibody (DAKO) and True-blot anti-rabbit antibody (eBiosciences). The HRP-coupled antibody directed against V5 (Invitrogen) and the anti-GST antibody (Rockland).

Cell Lines and their Treatments

293T, H1299 and U2OS cells were cultured in Dulbecco's modified Eagle's medium supplemented with 10% foetal calf serum (FCS) and 100 units/ml penicillin/streptomycin at 37° C. and 5% CO₂ in a humidified atmosphere. H1299 and 293T cells were transiently transfected by calcium-phosphate, U2OS cells were transfected with jetPEI (Biomol) according to the manufacturers recommendations.

### Immunoprecipitation and Western Blotting

Cells were washed twice with ice-cold phosphate-buffered saline (PBS) and lysed in NP-40 buffer (150 mM NaCl, 50 mM Tris pH 8, 5 mM EDTA, 1% NP-40, 1 mM phenylmethylsulfonyl fluoride). The protein extract was cleared by centrifugation at 13000 g at 4° C. for 15 min and the protein concentration was determined by the method of Bradford. 3 μl of the 9E10 anti-myc antibody, pre-coupled to Protein A-Agarose (Pierce), were added to 300 μg of the lysate and the mixture was incubated on a rotating wheel at 4° C. for 2 hours. The agarose was washed three times with NP-40 lysis buffer and resuspended in 1×SDS sample buffer (2% sodium dodecyl sulfate, 0.08 M Tris pH 6.8, 10% glycerol, 2% β-mercaptoethanol, 0.001% bromophenol blue).

For Western blotting, 50 μg of protein were mixed with an equal volume of 2×SDS sample buffer, heat denatured and loaded onto a SDS-10% PAGE gel. The proteins were transferred onto Immobilon-P blotting membrane (Millipore). Immunodetection was performed as described (Blattner et al., 2002).

### CoImmunoprecipitation

Coimmunoprecipitation of HDM2 and p53 from Cells:

JAR choriocarcinoma cells were seeded in 10 cm dishes at 3.6×106 cells/plate and were incubated the next day with the indicated concentrations of JNJ-#1, Nutlin-3 or the inactive enantiomer of Nutlin-3 for 1.5 hours.

Cells were washed twice with ice-cold phosphate-buffered saline (PBS) and lysed in Triton-X buffer (50 mM NaCl, 10 mM Tris pH 7.2, 5 mM EDTA, 1% Triton X-100). The protein extract was sonicated (for HDM2/p53 coIP), cleared by centrifugation at 13000 g at 4° C. for 15 min and the protein concentration was determined by the method of BCA/Pierce. The lysate (1 mg) was pre-cleared by adding 20 μl mouse IgG serum and 30 μl protein A-Agarose and incubating the mixture on a rotating wheel at 4° C. for 2 hours. Subsequently 10 μl of the 2A 10 anti-HDM2 antibody was added to the cleared lysate and rotated for 2 hours, and next 30 μl protein A-Agarose was added followed by rotating an additional 16 hours at 4° C. Immunoprecipitates were washed three times using Co-IP wash buffer (100 mM NaCl, 50 mM Tris pH 7.5, 1 mM EDTA, 0.1% Triton X-100, 5% glycerol). Immunoprecipitated HDM2 proteins were revealed using SMP-14 antibody (sc-965) and p53 protein was revealed as specified under Western Blot analysis.

Coimmunoprecipitation of HDM2 and Proteasomes from Cells:

Cells were washed twice with ice-cold phosphate-buffered saline (PBS) and lysed in NP-40 buffer (150 mM NaCl, 50 mM Tris pH 8, 5 mM EDTA, 1% NP-40, 1 mM phenylmethylsulfonyl fluoride). The protein extract was cleared by centrifugation at 13000 g at 4° C. for 15 min and the protein concentration was determined by the method of Bradford. 3 μl of the 9E10 anti-myc or anti-HDM2 antibody, pre-coupled to Protein A-Agarose (Pierce), were added to 1 mg of the lysate and the mixture was incubated on a rotating wheel at 4° C. for 1.5 hours. The agarose was washed three times with NP-40 lysis buffer. 40 μl 1×SDS sample buffer (2% sodium dodecyl sulfate, 0.08 M Tris pH 6.8, 10% glycerol, 2% β-mercaptoethanol, 0.001% bromophenol blue) were added and the samples were heat denatured before loading onto a SDS-10% PAGE gel. The proteins were transferred onto Immobilon-P blotting membrane (Millipore). Immunodetection was performed as described (Blattner et al., 2002).

### Coimmunoprecipitation of HDM2 and Proteasomes In Vitro:

100 ng GST-Hdm2 expressed in bacteria and 2 μl proteasomes (USbio) were preincubated with JNJ-#1 in 150 μl PWB buffer (50 mM Tris pH 7.5; 150 mM NaCl; 10% glycerol; 5 mM MgCl2; 2 mM ATP). Then HDM2 and proteasomes were combined and incubated for 30 mM at room temperature. 20 μl of the mixture were taken for input control and 3 μl of anti-C18 pre-coupled to 20 μl protein-A Agarose were added. The mixture was incubated for 1.5 hours on a rotating wheel at 4° C., washed 3× with PWB buffer and separated on a 10% SDS-PAGE gel.

### Western Blotting of the Lysates

U87 glioblastoma cells were incubated with the indicated concentrations of JNJ-#1 for 24 h. Total cell lysates were prepared and analyzed by SDS/PAGE. Levels of protein were detected using specific antibodies for p53 (DO-1, and pAb1801, Santa Cruz), p21^{waf1,cip} (BD Pharmingen), HDM2 (2A10, OP115 Calbiochem), E2F1 (sc-193, Santa Cruz), Rb (sc-102, Santa Cruz), pRb_Ser780 (9307L, Cell Signalling), anti-p73 (ab22045 Abcam), anti-p63 (sc-8431, Santa Cruz), cyclin G1 (sc-320, Santa Cruz), PIG3 (PC268, Calbiochem), MIC-1 (sc-10606, Santa Cruz). Actin protein levels (Ab-1, Oncogene Research products) were revealed as a control for equal loading. Protein-antibody complexes were visualized by chemiluminescence (Super Signal West Dura reagent, Pierce Chemical) and fluorescence (Odyssey) according to manufacturer's instructions.

### In Vitro p53 Degradation Assay

Flag-MDM2/p53 complexes were purified from High5 insect cells by a Flag-Agarose purification kit according to the recommendation of the supplier (Sigma).

0.2 μl partially purified E1 enzyme expressed in insect cells, 2 μl bacterial lysate of BL21 cells expressing UbCH5, 2 μl ubiquitin (5 μg/μl Sigma), 5 μl purified MDM2/p53 complexes, 1 μl Mg-ATP (100 mM) in 30 μl ubiquitin reaction buffer (25 mM Hepes pH 7.4; 10 mM NaCl; 3 mM MgCl2; 0.05% Triton X-100; 0.5 mM DTT). After 30 min reaction time, JNJ-#1 was added to a final concentration of 10 μM and incubated for 5 min at room temperature. 1 μl 26S proteasome (USbio) and 1 μl ATP (100 mM) were added and the reaction was incubated for 2.5 hours at 37° C. 1 μl of ATP (100 mM) was added and the reaction was incubated for further 2.5 hours. An equal volume of 2× sample buffer was added, the reaction was separated by a 8% SDS-PAGE gel and blotted onto Immobilon-P membrane.

### Limited Proteolysis

100 ng of GST fused to HdmX or fragments of HDM2 were incubated for 5 minutes with JNJ-#1, active or inactive nutlin, MG132 or DMSO, for control. Then 40 ng trypsin was added and the mixture was incubated for 15 min on ice. Proteolysis was stopped by addition of 2× sample buffer. Samples were loaded onto an 12 or 15% SDS-PAGE gel and HDM2 or HDMX were detected by Western blotting.

### Results

### JNJ-#1 Binds to HDM2.

To investigate binding of JNJ-#1, one of the compounds shown to be an HDM2 antagonists in PCT publication WO2006/032631, to HDM2, we determined its influence on proteolysis of HDM2 by the proteolytic enzyme Trypsin. We therefore incubated bacterially expressed HDM2, fused to GST, in the presence or absence of JNJ-#1 with Trypsin under conditions were proteolysis of HDM2 was incomplete (limited proteolysis).

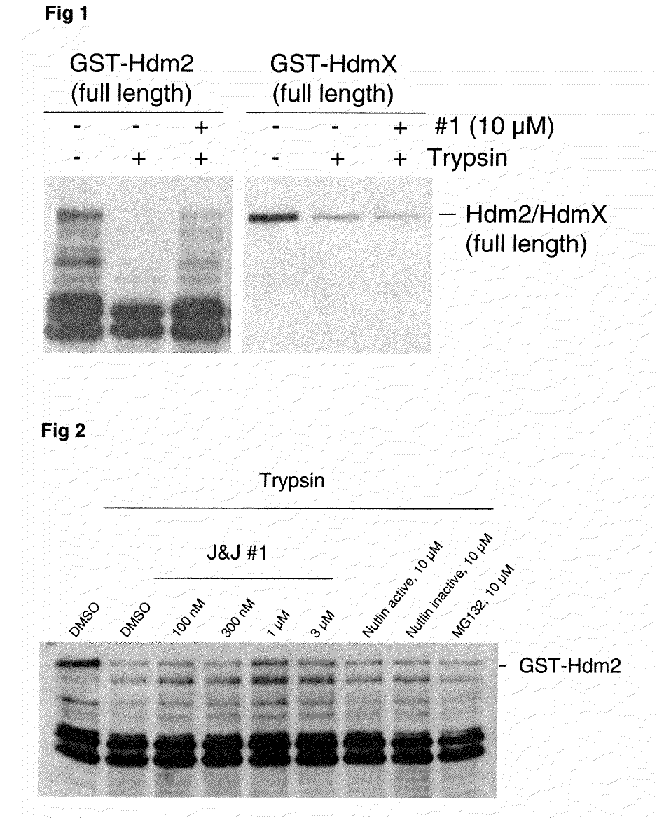

As we show in FIG. 1, JNJ-#1 strongly reduced proteolysis of full length HDM2, but not proteolysis of the HDM2 family member HDMX, which is a close homologue of HDM2.

JNJ-#1 Interferes with HDM2 Proteolysis at Doses as Low as 100 nM.

We next determined the dose of JNJ-#1 that is required for the interference with HDM2 proteolysis. We incubated bacterially expressed HDM2 fused to GST in the presence of increasing concentrations of JNJ-#1 with Trypsin. To investigate the specificity of the JNJ-#1 compound, we also incubated GST-HDM2 with active and inactive nutlin as well as with the proteasome inhibitor MG132. As we show in FIG. 2, proteolysis of HDM2 was inhibited by JNJ-#1 already at doses as low as 100 nM. Increasing the dose of JNJ-#1 up to 1 μM further reduced proteolysis of HDM2, and no further reduction was observed at 3 μM. In contrast, incubation of GST-HDM2 with a dose of 10 μM active or inactive nutlin affected proteolysis only weakly and incubation of GST-HDM2 with 10 μM of MG132 had no affect.

JNJ-#1 Induces p53 and Activates Downstream Signalling in Tumor Cells.

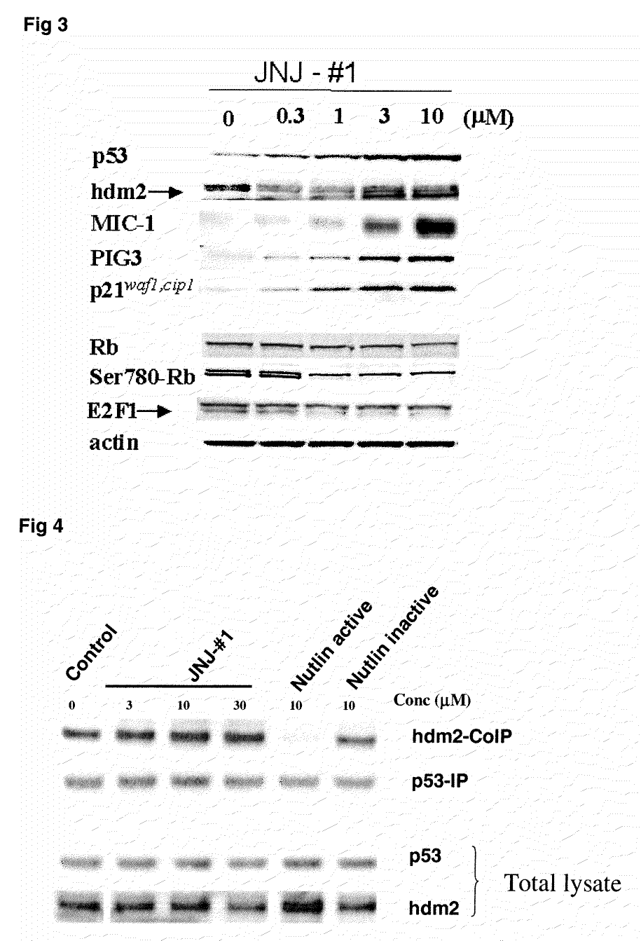

JNJ-#1 has been identified to bind and change conformation of hDM2. JNJ-#1 was first investigated as to whether the compound affects the expression of HDM2 binding partners such as p53 and E2F1 and their downstream signaling molecules. U-87 MG glioblastoma cells were incubated with JNJ-#1 for 24 hours, and as shown in FIG. 3 JNJ-#1 induced p53 starting at 1 μM, further increasing up to 10 μM. The expression of E2F-1, which is essential for S-phase progression was dramatically decreased. In parallel to the p53 induction, a clear increase in the downstream genes p21^{waf1, cip1}, MIC-1, and PIG3 was observed after 24 hours of incubation, starting at 1 μM, but reaching its maximal induction at 10 μM. Hitself, which is also transcriptionally induced by p53, was not detectable under control conditions, but clearly induced by

JNJ-#1 at 1 to 10 μM. Induction of the cyclin dependent kinase (cdk) inhibitor p21^{waf1, cip1} is expected to result in decrease in cdk mediated phosphorylation of the Retinoblastoma (Rb) tumor suppressor protein, which results in a block of E2F-1 activity and thereby G1 cell cycle arrest. As shown in FIG. 3, indeed a potent decrease in Ser780-Rb phosphorylation was observed already at 1 μM, which parallels p21^{waf1, cip1} induction.

### JNJ-#1 Does not Prevent Binding of HDM2 to p53 in Jar Choriocarcinoma Cells

Since JNJ-#1 was identified as an hDM2 antagonist, and induces p53 protein levels, we subsequently investigated whether the compound displaces p53 from HDM2, thereby preventing p53 degradation. For this purpose we utilised JAR choriocarcinoma cells, which have high HDM2 expression levels due to a gene amplification. As illustrated in FIG. 4, when endogenous HDM2 was immunoprecipitated from JAR cells, the amount of p53 attached was increased in the presence of JNJ-#1. The positive control HDM2 antagonist Nutlin-3, which is known to bind the N-terminal pocket of HDM2 efficiently displaced p53 form the HDM2 protein, while the inactive enantiomer of Nutlin-3 had no effect. These data indicate that JNJ-#1 affect HDM2 function through a novel mechanism of action.

### JNJ-#1 Does not Inhibit p53 Ubiquitination in U2OS Cells

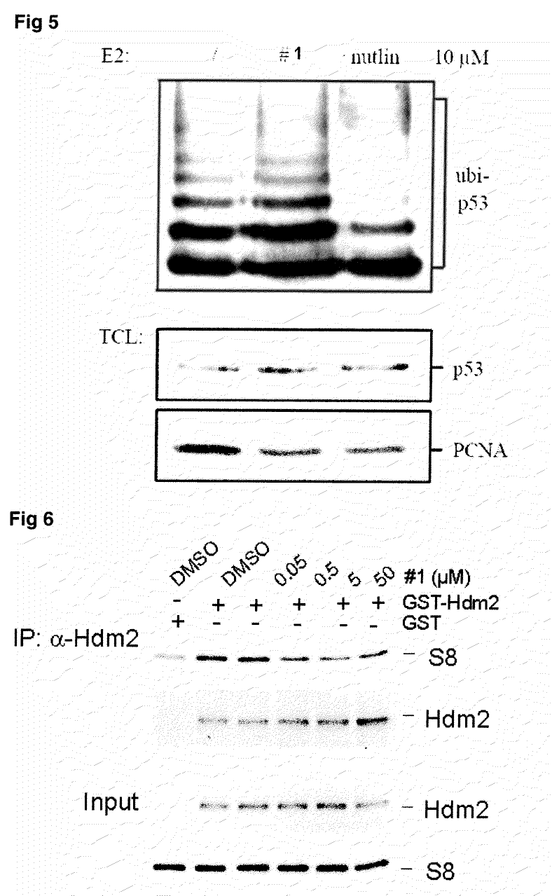

JNJ-#1 leads to the accumulation of p53. One very likely mechanism would be that JNJ-#1 prevents p53 ubiquitylation by affecting HDM2 ubiquitin ligase acetivity. To test this option, we performed an cellular ubiquitylation assay in the presence of the compound. As shown in FIG. 5, ubiquitylation of p53 was strongly reduced in the presence of Nutlin-3 while it was not affected at all by JNJ-#1.

### JNJ-#1 Prevents Binding of HDM2 to the Proteasome in a Dose Dependent Manner.

We previously observed that HDM2 associates with the proteasome and we speculated that this interaction might impact on p53 degradation. To investigate the impact of JNJ-#1 on the interaction of HDM2 with the proteasome, we incubated bacterially expressed HDM2 fused to GST and proteasomes with increasing doses of JNJ-#1. We then mixed HDM2 and the proteasomes and incubated the mixture for 30 min at room temperature prior to immune-precipitation of HDM2. We separated the complexes by SDS-PAGE and determined the relative amount of proteasomes associated with HDM2 by Western blotting.

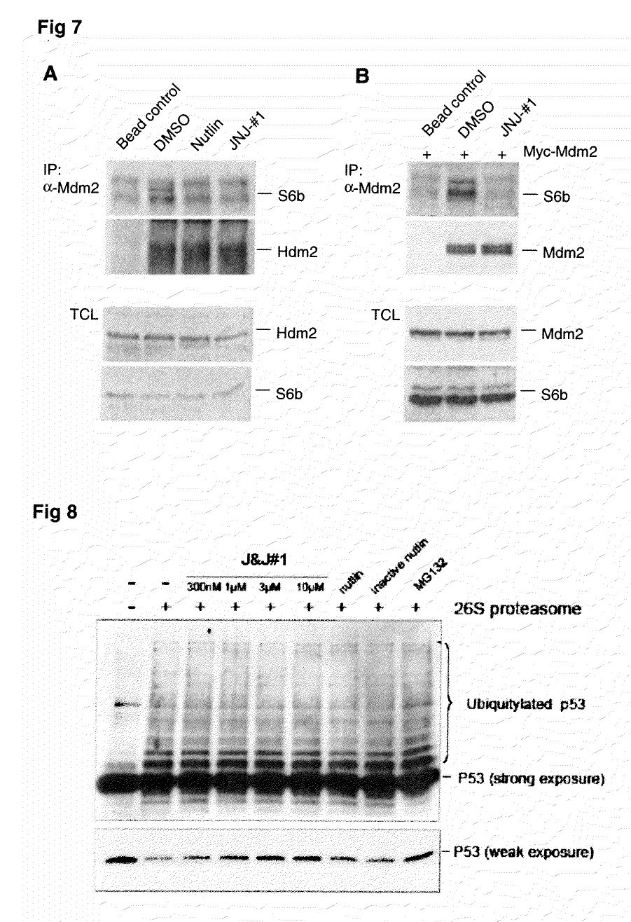

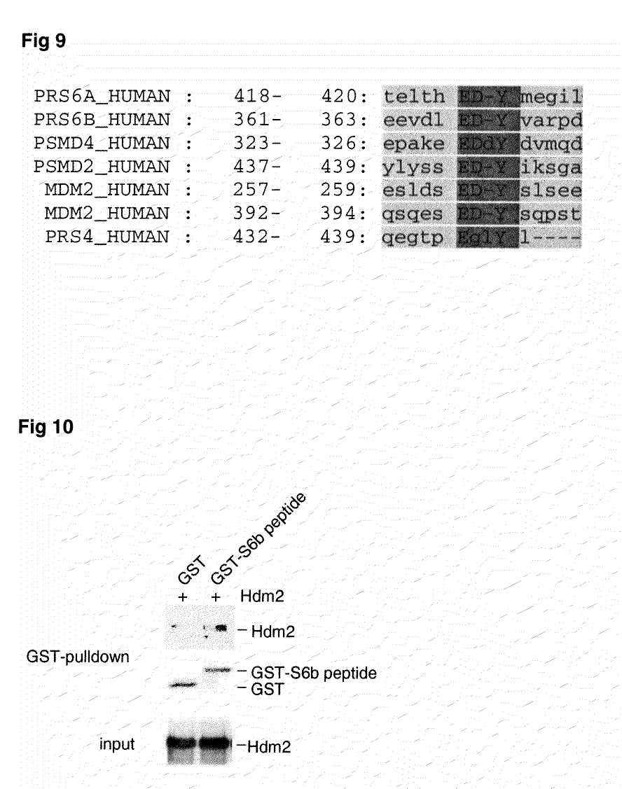

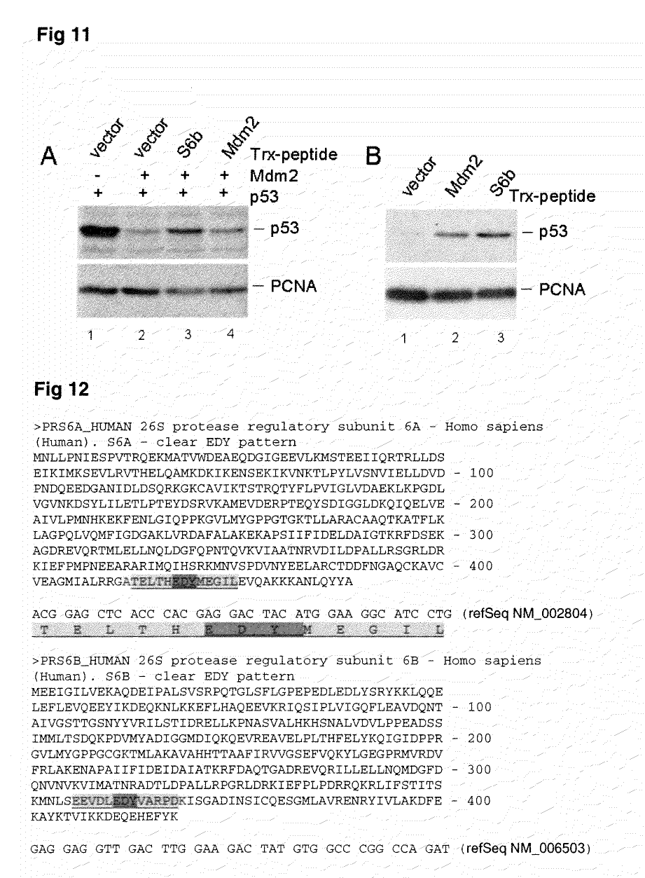

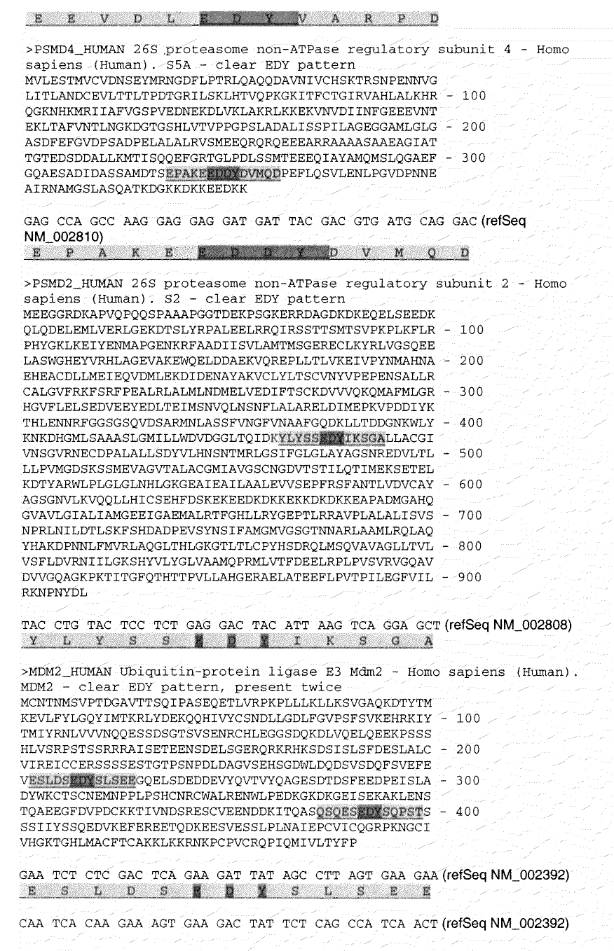

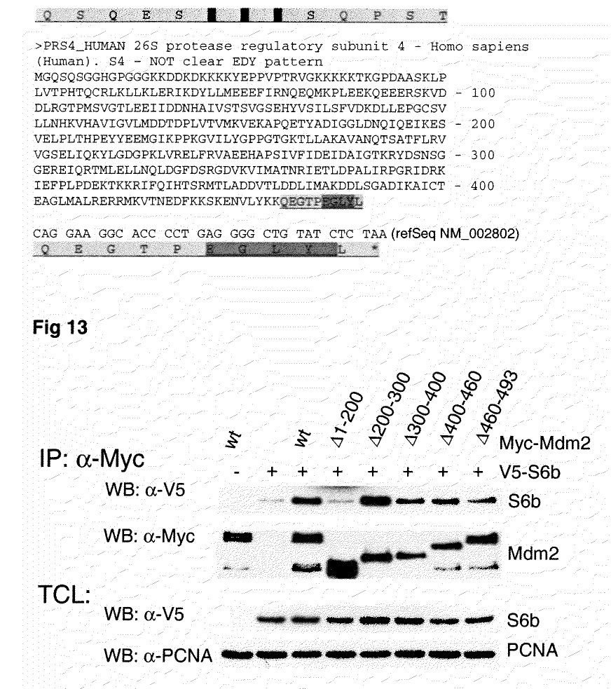

As we show in FIG. 6, GST alone bound only weakly to proteasomes, as visualised by the S8 subunit. When we included HDM2 in the assay, a significantly higher amount of proteasomes co-precipitated with GST-HDM2 than with GST-alone. The addition of JNJ-#1 at a concentration as low as 50 nM did not affect this association. However, when we raised the concentration of JNJ-#1 to 500 nM, the association of HDM2 with proteasomes was strongly reduced. Increasing the dose of JNJ-#1 to 5 μM further reduced the association of HDM2 with the proteasome.

### JNJ-#1 Prevents Binding of HDM2 to the Proteasome in Cells.

We next determined whether JNJ-#1 also prevented the association of HDM2 with the proteasome in cells. We incubated cells with 10 μM JNJ-#1, with 10 μM nutlin or with DMSO for control, immunoprecipitated HDM2 with an anti-HDM2 antibody and determined the relative amount of associated S6b by Western blotting. The proteasome subunit S6b co-precipitated with HDM2 in the absence of JNJ-#1. However, in the presence of 10 μM JNJ-#1 or in the presence of nutlin, the association of HDM2 with S6b was no longer detectable (data not shown). To confirm the result, we repeated the experiment with overexpressed Myc-tagged MDM2. We transfected Myc-MDM2 in 293 T cells. Twenty-four hours after transfection, 10 μM JNJ-#1 or DMSO for control were added to the cells. After 1.5 hour incubation time, Myc-MDM2 was precipitated using the anti-Myc-antibody 9E10. Associating proteasomes were determined by Western blotting. Like with endogenous HDM2, the proteasome co-precipitated with Myc-tagged MDM2. JNJ-#1 again prevented the interaction completely (FIG. 7)

### JNJ-#1 Prevents Degradation of p53 In Vitro.

To determine whether the reduction in the association of HDM2 with the proteasome in the presence of JNJ-#1 influences p53 degradation, we employed an in vitro degradation assay. We mixed p53 and HDM2 that were expressed in baculoviruses with ubiquitin, 26S proteasomes and E1 and E2 enzymes. In the absence of JNJ-#1, p53 was quickly degraded. However, when we performed the reaction in the presence of JNJ-#1, degradation of p53 was completely abrogated (FIG. 8).

### HDM2/MDM2 Share a Sequence Motif Comprising the Amino Acids EDY

To determine the interaction site with the proteasome on MDM2, we transfected 293T cells with a series of MDM2 deletion mutants together with a plasmid encoding the S6b protein of the proteasome. After co-transfection of S6b with a mutant of MDM2 lacking the central domain, binding of Mdm2 to S6b was significantly enhanced in comparison to the binding of wild type MDM2 to S6b (FIG. 13). From this result, we concluded that a sequence motif in the central domain interferes with the binding of MDM2 to the proteasome. Therefore, the central domain might reduce the interaction of MDM2 with the proteasome by binding to a sequence of MDM2 that is also able to associate with the proteasome, so a sequence motif should be common to MDM2 and the proteasome. In search for such a sequence, we aligned the central domain of MDM2 with several subunits of the proteasome. By this, we identified a three amino acid sequence, the EDY motif, that is present in MDM2 and in several subunits of the 26S proteasome (FIG. 9, FIG. 12).

A peptide Containing the EDY Motif Associates with MDM2

We determined whether MDM2 is able to associates with the EDY motif of the proteasome by performing a GST-pulldown assay using a GST-fusion protein comprising a peptide from the S6b protein encompassing the EDY sequence and MDM2 expressed from baculovirus. As we show in FIG. 10, to our surprise MDM2 clearly associated with the GST-fusion protein encompassing the S6b-derived peptide, but not with GST alone.

Overexpression of the EDY Motif of HDM2/MDM2 or S6b Interferes with p53 Degradation

If the EDY-motif is mediating the association of HDM2 with the proteasome, a peptide containing the EDY sequence should compete with the proteasome for binding to HDM2. Moreover, if the association of HDM2 with the proteasome is important for p53 degradation, p53 should accumulate after overexpression of such a peptide. In FIG. 11, we show that this is indeed the case. When we transfected a thioredoxin-insertion construct where the sequence encompassing the EDY motif of S6b or HDM2 was expressed in an outer loop of the thioredoxin protein into H1299 cells, p53 degradation by MDM2 was strongly reduced. Likewise, when we expressed the Thioredoxin-insertion proteins in U2OS cells, degradation of endogenous p53 was almost completely abrogated.

1. A method to identify a compound that affects binding of HDM2 to the proteasome.

2. A method as claimed in claim 1, said method comprising:
a) contacting the compound to be tested with a HDM2 protein, a related protein or a protein binding fragment thereof and
b) determining whether said compound affects the proteolysis of a HDM2 binding protein by the ubiquitin-proteasome proteolysis pathway.

3. The method as claimed in claim 1 or 2 wherein the HDM2 binding protein is p53.

4. The method as claimed in claim 1 or 2 wherein the HDM2 binding protein is other than p53.

5. A method as claimed in claim 1, said method comprising:
a) incubating the proteasome subunit or protein binding fragments thereof with labelled HDM2, labelled related proteins or labelled protein binding fragments thereof,
b) adding the test compound to the incubation mixture, and
c) measuring the effect of the test compound on the amount of labelled HDM2, labelled related proteins or labelled protein binding fragments thereof bound to the proteasome subunit or protein binding fragments thereof.

6. A method as claimed in claim 1, said method comprising:
a) incubating the labelled proteasome subunit or labelled protein binding fragments thereof with HDM2, related proteins or protein binding fragments thereof,
b) adding the test compound to the incubation mixture, and
c) measuring the effect of the test compound on the amount of labelled proteasome subunit or labelled protein binding fragments thereof bound to HDM2, related proteins or protein binding fragments thereof.

7. A method as claimed in claim 1, said method comprising:
a) probing the proteasome subunit or protein binding fragments thereof with HDM2, related proteins or protein binding fragments thereof,
b) identifying contacting atoms in the binding site of the proteasome subunit or protein binding fragments thereof that interact with HDM2, related proteins or protein binding fragments thereof or vice versa,
c) designing test compounds that interact with the atoms identified in (b), and
d) contacting said designed test compound with a proteasome subunit, a protein binding fragment thereof, HDM2, related proteins or protein binding fragments thereof to measure the capability of said compound to modulate the interaction between HDM2 and the proteasome or to modulate ubiquitinin-proteasome proteolysis.

8. A method as claimed of claim 1 wherein the protein binding fragment of HDM2 is selected from a group of polypeptide sequences or a member of a group of polypeptide sequences comprising:
a) amino acid sequences SEQ ID NO:11 or SEQ ID NO 12 or homologs thereof wherein said homologs have at least 70, 80, 85, 90, 95, 97, 98 or 99% identity to SEQ ID NO:11 or SEQ ID NO 12,
b) amino acids 252-264 (SEQ ID No:11) or 387-399 (SEQ ID No:12) of HDM2 or homologs thereof wherein said homologs have at least 70, 80, 85, 90, 95, 97, 98 or 99% identity to SEQ ID NO:11 or SEQ ID NO:1, or
c) amino acids 257-259 or 392-394 of HDM2 (SEQ ID NO:5) or homologs thereof wherein said homologs have at least 70, 80, 85, 90, 95, 97, 98 or 99% sequence identity to SEQ ID No:5.

9. A method as claimed of claim 1 wherein the protein binding fragment of the proteasome unit is selected from a group of polypeptide sequences or a member of a group of polypeptide sequences comprising:
a) amino acid sequences SEQ ID No:7, SEQ ID No:8, SEQ ID No:9, SEQ ID No:10 or SEQ ID No:13 or homologs thereof wherein said homologs have at least 70, 80, 85, 90, 95, 97, 98 or 99% identity to any one of SEQ ID No:7, SEQ ID No:8, SEQ ID No:9, SEQ ID No:10 or SEQ ID No:13,
b) amino acids 413-425 (SEQ ID NO:7) of S6A, amino acids 356-368 (SEQ ID NO:8) of S6B, amino acids 318-331 (SEQ ID NO:9) of S5A, amino acids 432-444 (SEQ ID NO:10) of S2 or amino acids 431-440 (SEQ ID NO:13) of S4 or homologs thereof wherein said homologs have at least 70, 80, 85, 90, 95, 97, 98 or 99% sequence identity to SEQ ID NO:7, SEQ ID NO:8, SEQ ID NO:9, SEQ ID NO:10 or SEQ ID No:13, or
c) amino acids 418-420 of S6A (SEQ ID NO:1), amino acids 418-420 of S6A (SEQ ID NO:2), amino acids 361-363 of S6B (SEQ ID NO:3), amino acids 323-326 of S5A (SEQ ID NO:4), amino acids 437-439 of S2 (SEQ ID NO:6) or amino acids 436-439 of S4 (SEQ ID NO:13) or homologs thereof wherein said homologs have at least 70, 80, 85, 90, 95, 97, 98 or 99% sequence identity to SEQ ID NO:1, SEQ ID NO:2, SEQ ID NO:3, SEQ ID NO:4 or SEQ ID No:6.

10. The protein binding fragments of HDM2 as defined in claim 8 or the protein binding fragments of the proteasome subunits as defined in claim 9, for use as a medicine.

11. A group of nucleotide sequences or a member of a group of nucleotide sequences coding for the polypeptides as defined in claim 8 comprising:
a) nucleotide sequences SEQ ID NO:18 or SEQ ID NO 19 or homologs thereof, wherein said homologs have at least 70, 80, 85, 90, 95, 97, 98 or 99% identity to SEQ ID NO:18 or SEQ ID NO 19,
b) nucleotide sequences 1050-1088 (SEQ ID No:18) or 1455-1493 (SEQ ID No:19) of HDM2 or homologs thereof wherein said homologs have at least 70, 80, 85, 90, 95, 97, 98 or 99% identity to SEQ ID NO:18 or SEQ ID NO:19, or
c) nucleotide sequences 16-24 of SEQ ID NO:18 or SEQ ID NO 19 or homologs thereof wherein said homologs have at least 70, 80, 85, 90, 95, 97, 98 or 99% sequence identity to SEQ ID No:25,
for use as a medicine.

12. A group of nucleotide sequences or a member of a group of nucleotide sequences coding for the polypeptides as defined in claim 9 comprising:
a) the ED(X)Y nucleotide sequence of proteasome subunits S6a, S6b, S5a, S4 or S2 or a homologue thereof wherein said homolog has at least 70, 80, 85, 90, 95, 97, 98 or 99% identity to SEQ ID NO:14, SEQ ID NO:15, SEQ ID NO:16, SEQ ID NO:17 or SEQ ID NO 20.
b) the nucleotides sequence 1431-1469 of SEQ ID No:21, 1103-1141 of SEQ ID No:22, 1014-1055 of SEQ ID No:23, 1327-1365 of SEQ ID No:24 or 1339-1371 of SEQ ID No:26 of proteasome subunits S6a, S6b, S5a, S4 or S2 or a homolog thereof wherein said homolog has at least 70, 80, 85, 90, 95, 97, 98 or 99% identity to SEQ ID NO:21, ID NO:22, ID NO:23, ID NO:24 or SEQ ID NO:16.
c) the nucleotides sequence 1-39 of SEQ ID NO:14, SEQ ID NO:15 or SEQ ID NO:17, the nucleotides sequence 1-42 of SEQ ID NO:16 or the nucleotides sequence 1-33 of SEQ ID NO:20 or a homolog thereof wherein said homolog has at least 70, 80, 85, 90, 95, 97, 98 or 99% sequence identity to SEQ ID NO:14, SEQ ID NO:15, SEQ ID NO:16, SEQ ID NO:17 or SEQ ID NO 20.
d) the nucleotides sequence 16-24 of SEQ ID NO:14, SEQ ID NO:15 or SEQ ID NO:17, the nucleotides sequence 16-27 of SEQ ID NO:16 or SEQ ID NO:20 or a homolog thereof wherein said homolog has at least 70, 80, 85, 90, 95, 97, 98 or 99% sequence identity to SEQ ID NO:14, SEQ ID NO:15, SEQ ID NO:16, SEQ ID NO:17 or SEQ ID NO 20
for use as a medicine.

13. Use of a polypeptide sequence as defined in claim 7 or 8 or of a nucleotides sequence as defined in claim 10 or 11, for the manufacture of a medicament for the treatment of cancer and leukemia.


SEQUENCE LISTING (26 sequences)

SEQ ID NO: 1 - protein, 439 aa, Homo sapiens
MNLLPNIESPVTRQEKMATVWDEAEQDGIGEEVLKMSTEEIIQRTRLLDSEIKIMKSEVLRVTHELQAMKDKIKENSEKIKVNKTLPYLVSNVIELLDVDPNDQEEDGANIDLDSQRKGKCAVIKTSTRQTYFLPVIGLVDAEKLKPGDLVGVNKDSYLILETLPTEYDSRVKAMEVDERPTEQYSDIGGLDKQIQELVEAIVLPMNHKEKFENLGIQPPKGVLMYGPPGTGKTLLARACAAQTKATFLKLAGPQLVQMFIGDGAKLVRDAFALAKEKAPSIIFIDELDAIGTKRFDSEKAGDREVQRTMLELLNQLDGFQPNTQVKVIAATNRVDILDPALLRSGRLDRKIEFPMPNEEARARIMQIHSRKMNVSPDVNYEELARCTDDFNGAQCKAVCVEAGMIALRRGATELTHEDYMEGILEVQAKKKANLQYYA

SEQ ID NO: 2 - protein, 418 aa, Homo sapiens
MEEIGILVEKAQDEIPALSVSRPQTGLSFLGPEPEDLEDLYSRYKKLQQELEFLEVQEEYIKDEQKNLKKEFLHAQEEVKRIQSIPLVIGQFLEAVDQNTAIVGSTTGSNYYVRILSTIDRELLKPNASVALHKHSNALVDVLPPEADSSIMMLTSDQKPDVMYADIGGMDIQKQEVREAVELPLTHFELYKQIGIDPPRGVLMYGPPGCGKTMLAKAVAHHTTAAFIRVVGSEFVQKYLGEGPRMVRDVFRLAKENAPAIIFIDEIDAIATKRFDAQTGADREVQRILLELLNQMDGFDQNVNVKVIMATNRADTLDPALLRPGRLDRKIEFPLPDRRQKRLIFSTITSKMNLSEEVDLEDYVARPDKISGADINSICQESGMLAVRENRYIVLAKDFEKAYKTVIKKDEQEHEFYK

SEQ ID NO: 3 - protein, 377 aa, Homo sapiens
MVLESTMVCVDNSEYMRNGDFLPTRLQAQQDAVNIVCHSKTRSNPENNVGLITLANDCEVLTTLTPDTGRILSKLHTVQPKGKITFCTGIRVAHLALKHRQGKNHKMRIIAFVGSPVEDNEKDLVKLAKRLKKEKVNVDIINFGEEEVNTEKLTAFVNTLNGKDGTGSHLVTVPPGPSLADALISSPILAGEGGAMLGLGASDFEFGVDPSADPELALALRVSMEEQRQRQEEEARRAAAASAAEAGIATTGTEDSDDALLKMTISQQEFGRTGLPDLSSMTEEEQIAYAMQMSLQGAEFGQAESADIDASSAMDTSEPAKEEDDYDVMQDPEFLQSVLENLPGVDPNNEAIRNAMGSLASQATKDGKKDKKEEDKK

SEQ ID NO: 4 - protein, 908 aa, Homo sapiens
MEEGGRDKAPVQPQQSPAAAPGGTDEKPSGKERRDAGDKDKEQELSEEDKQLQDELEMLVERLGEKDTSLYRPALEELRRQIRSSTTSMTSVPKPLKFLRPHYGKLKEIYENMAPGENKRFAADIISVLAMTMSGERECLKYRLVGSQEELASWGHEYVRHLAGEVAKEWQELDDAEKVQREPLLTLVKEIVPYNMAHNAEHEACDLLMEIEQVDMLEKDIDENAYAKVCLYLTSCVNYVPEPENSALLRCALGVFRKFSRFPEALRLALMLNDMELVEDIFTSCKDVVVQKQMAFMLGRHGVFLELSEDVEEYEDLTEIMSNVQLNSNFLALARELDIMEPKVPDDIYKTHLENNRFGGSGSQVDSARMNLASSFVNGFVNAAFGQDKLLTDDGNKWLYKNKDHGMLSAAASLGMILLWDVDGGLTQIDKYLYSSEDYIKSGALLACGIVNSGVRNECDPALALLSDYVLHNSNTMRLGSIFGLGLAYAGSNREDVLTLLLPVMGDSKSSMEVAGVTALACGMIAVGSCNGDVTSTILQTIMEKSETELKDTYARWLPLGLGLNHLGKGEAIEAILAALEVVSEPFRSFANTLVDVCAYAGSGNVLKVQQLLHICSEHFDSKEKEEDKDKKEKKDKDKKEAPADMGAHQGVAVLGIALIAMGEEIGAEMALRTFGHLLRYGEPTLRRAVPLALALISVSNPRLNILDTLSKFSHDADPEVSYNSIFAMGMVGSGTNNARLAAMLRQLAQYHAKDPNNLFMVRLAQGLTHLGKGTLTLCPYHSDRQLMSQVAVAGLLTVLVSFLDVRNIILGKSHYVLYGLVAAMQPRMLVTFDEELRPLPVSVRVGQAVDVVGQAGKPKTITGFQTHTTPVLLAHGERAELATEEFLPVTPILEGFVILRKNPNYDL

SEQ ID NO: 5 - protein, 491 aa, Homo sapiens
MCNTNMSVPTDGAVTTSQIPASEQETLVRPKPLLLKLLKSVGAQKDTYTMKEVLFYLGQYIMTKRLYDEKQQHIVYCSNDLLGDLFGVPSFSVKEHRKIYTMIYRNLVVVNQQESSDSGTSVSENRCHLEGGSDQKDLVQELQEEKPSSSHLVSRPSTSSRRRAISETEENSDELSGERQRKRHKSDSISLSFDESLALCVIREICCERSSSSESTGTPSNPDLDAGVSEHSGDWLDQDSVSDQFSVEFEVESLDSEDYSLSEEGQELSDEDDEVYQVTVYQAGESDTDSFEEDPEISLADYWKCTSCNEMNPPLPSHCNRCWALRENWLPEDKGKDKGEISEKAKLENSTQAEEGFDVPDCKKTIVNDSRESCVEENDDKITQASQSQESEDYSQPSTSSSIIYSSQEDVKEFEREETQDKEESVESSLPLNAIEPCVICQGRPKNGCIVHGKTGHLMACFTCAKKLKKRNKPCPVCRQPIQMIVLTYFP

SEQ ID NO: 6 - protein, 440 aa, Homo sapiens
MGQSQSGGHGPGGGKKDDKDKKKKYEPPVPTRVGKKKKKTKGPDAASKLPLVTPHTQCRLKLLKLERIKDYLLMEEEFIRNQEQMKPLEEKQEEERSKVDDLRGTPMSVGTLEEIIDDNHAIVSTSVGSEHYVSILSFVDKDLLEPGCSVLLNHKVHAVIGVLMDDTDPLVTVMKVEKAPQETYADIGGLDNQIQEIKESVELPLTHPEYYEEMGIKPPKGVILYGPPGTGKTLLAKAVANQTSATFLRVVGSELIQKYLGDGPKLVRELFRVAEEHAPSIVFIDEIDAIGTKRYDSNSGGEREIQRTMLELLNQLDGFDSRGDVKVIMATNRIETLDPALIRPGRIDRKIEFPLPDEKTKKRIFQIHTSRMTLADDVTLDDLIMAKDDLSGADIKAICTEAGLMALRERRMKVTNEDFKKSKENVLYKKQEGTPEGLYL

SEQ ID NO: 7 - protein, 13 aa, Homo sapiens
TELTHEDYMEGIL

SEQ ID NO: 8 - protein, 13 aa, Homo sapiens
EEVDLEDYVARPD

SEQ ID NO: 9 - protein, 14 aa, Homo sapiens
EPAKEEDDYDVMQD

SEQ ID NO: 10 - protein, 13 aa, Homo sapiens
YLYSSEDYIKSGA

SEQ ID NO: 11 - protein, 13 aa, Homo sapiens
ESLDSEDYSLSEE

SEQ ID NO: 12 - protein, 13 aa, Homo sapiens
QSQESEDYSQPST

SEQ ID NO: 13 - protein, 10 aa, Homo sapiens
QEGTPEGLYL

SEQ ID NO: 14 - DNA, 39 bp, Homo sapiens
acggagctcacccacgaggactacatggaaggcatcctg

SEQ ID NO: 15 - DNA, 39 bp, Homo sapiens
gaggaggttgacttggaagactatgtggcccggccagat

SEQ ID NO: 16 - DNA, 42 bp, Homo sapiens
gagccagccaaggaggaggatgattacgacgtgatgcaggac

SEQ ID NO: 17 - DNA, 39 bp, Homo sapiens
tacctgtactcctctgaggactacattaagtcaggagct

SEQ ID NO: 18 - DNA, 39 bp, Homo sapiens
gaatctctcgactcagaagattatagccttagtgaagaa

SEQ ID NO: 19 - DNA, 39 bp, Homo sapiens
caatcacaagaaagtgaagactattctcagccatcaact

SEQ ID NO: 20 - DNA, 33 bp, Homo sapiens
caggaaggcacccctgaggggctgtatctctaa

SEQ ID NO: 21 - DNA, 1618 bp, Homo sapiens
gtggccaggggttggtctttggaatcggagaggcagattcgctgctccgcagcacggccggagctggtcgggtcaagagtcgggatttgtggggagaggttttccactggtcaagagaaggctttaagaaagacggtattaatctcccgttgcggctcccgcctggtcccatcttctgcccgctcctccaggaaatgaatctgctgccgaatattgagagtccagtgactcggcaggagaagatggcgaccgtgtgggatgaggccgagcaagatggaattggggaggaggtgctcaagatgtccacggaggagatcatccagcgcacacggctgctggacagtgagatcaagatcatgaagagtgaagtgttgagagtcacccatgagctccaagccatgaaggacaagataaaagagaacagtgagaaaatcaaagtgaacaagaccctgccgtaccttgtctccaacgtcatcgagctcctggatgttgatcctaatgaccaagaggaggatggtgccaatattgacctggactcccagaggaagggcaagtgtgctgtgatcaaaacctctacacgacagacgtacttccttcctgtgattgggttggtggatgctgaaaagctaaagccaggagacctggtgggtgtgaacaaagactcctatctgatcctggagacgctgcccacagagtatgactcgcgggtgaaggccatggaggtagacgagaggcccacggagcaatacagtgacattgggggtttggacaagcagatccaggagctggtggaggccattgtcttgccaatgaaccacaaggagaagtttgagaacttggggatccaacctccaaaaggggtgctgatgtatgggcccccagggacggggaagaccctcctggcccgggcctgtgccgcacagactaaggccaccttcctaaagctggctggcccccagctggtgcagatgttcattggagatggtgccaagctagtccgggatgcctttgccctggccaaggagaaagcgccctctatcatcttcattgatgagttggatgccatcggcaccaagcgctttgacagtgagaaggctggggaccgggaggtgcagaggacaatgctggagcttctgaaccagctggatggcttccagcccaacacccaagttaaggtaattgcagccacaaacagggtggacatcctggaccccgccctcctccgctcgggccgccttgaccgcaagatagagttcccgatgcccaatgaggaggcccgggccagaatcatgcagatccactcccgaaagatgaatgtcagtcctgacgtgaactacgaggagctggcccgctgcacagatgacttcaatggggcccagtgcaaggctgtgtgtgtggaggcgggcatgatcgcactgcgcaggggtgccacggagctcacccacgaggactacatggaaggcatcctggaggtgcaggccaagaagaaagccaacctacaatactacgcctagggcacacaggccagccccagtctcacggctgaagtgcgcaataaaagatggtttagggtccctgccaaaaaaaaaaaaaaaaaaaaaaaaaaaaaaaaaaaaaa

SEQ ID NO: 22 - DNA, 1450 bp, Homo sapiens
gatcatcccaggccacacagaggccggcttggtcactatggaggagataggcatcttggtggagaaggctcaggatgagatcccagcactgtccgtgtcccggccccagaccggcctgtccttcctgggccctgagcctgaggacctggaggacctgtacagccgctacaagaagctgcagcaagagctggagttcctggaggtgcaggaggaatacatcaaagatgagcaaaagaacctgaaaaaggaatttctccatgcccaggaggaggtgaagcgaatccaaagcatcccgctggtcatcggacaatttctggaggctgtggatcagaatacagccatcgtgggctctaccacaggctccaactattatgtgcgcatcctgagcaccatcgatcgggagctgctcaagcccaacgcctcagtggccctccacaagcacagcaatgcactggtggacgtgctgccccccgaagccgacagcagcatcatgatgctcacctcagaccagaagccagatgtgatgtacgcggacatcggaggcatggacatccagaagcaggaggtgcgggaggccgtggagctcccgctcacgcatttcgagctctacaagcagatcggcatcgatcccccccgaggcgtcctcatgtatggcccacctggctgtgggaagaccatgttggcaaaggcggtggcacatcacacaacagctgcattcatccgggtcgtgggctcggagtttgtacagaagtatctgggtgagggcccccgcatggtccgggatgtgttccgcctggccaaggagaatgcacctgccatcatcttcatagacgagattgatgccatcgccaccaagagattcgatgctcagacaggggccgacagggaggttcagaggatcctgctggagctgctgaatcagatggatggatttgatcagaatgtcaatgtcaaggtaatcatggccacaaacagagcagacaccctggatccggccctgctacggccaggacggctggaccgtaaaattgaatttccacttcctgaccgccgccagaagagattgattttctccactatcactagcaagatgaacctctctgaggaggttgacttggaagactatgtggcccggccagataagatttcaggagctgatattaactccatctgtcaggagagtggaatgttggctgtccgtgaaaaccgctacattgtcctggccaaggacttcgagaaagcatacaagactgtcatcaagaaggacgagcaggagcatgagttttacaagtgacccttcccttccctccaccacaccactcaggggctggggcttctctcgcacccccagcacctctgtcccaaaacctcattcccttttttctttacccaggattggtttcttcaataaatagataagatcgaatccatttaaaaaaaaaaaaaaaaa

SEQ ID NO: 23 - DNA, 1332 bp, Homo sapiens
aattggaggagttgttgttaggccgtcccggagacccggtcgggagggaggaaggtggcaagatggtgttggaaagcactatggtgtgtgtggacaacagtgagtatatgcggaatggagacttcttacccaccaggctgcaggcccagcaggatgctgtcaacatagtttgtcattcaaagacccgcagcaaccctgagaacaacgtgggccttatcacactggctaatgactgtgaagtgctgaccacactcaccccagacactggccgtatcctgtccaagctacatactgtccaacccaagggcaagatcaccttctgcacgggcatccgcgtggcccatctggctctgaagcaccgacaaggcaagaatcacaagatgcgcatcattgcctttgtgggaagcccagtggaggacaatgagaaggatctggtgaaactggctaaacgcctcaagaaggagaaagtaaatgttgacattatcaattttggggaagaggaggtgaacacagaaaagctgacagcctttgtaaacacgttgaatggcaaagatggaaccggttctcatctggtgacagtgcctcctgggcccagtttggctgatgctctcatcagttctccgattttggctggtgaaggtggtgccatgctgggtcttggtgccagtgactttgaatttggagtagatcccagtgctgatcctgagctggccttggcccttcgtgtatctatggaagagcagcggcagcggcaggaggaggaggcccggcgggcagctgcagcttctgctgctgaggccgggattgctacgactgggactgaagactcagacgatgccctgctgaagatgaccatcagccagcaagagtttggccgcactgggcttcctgacctaagcagtatgactgaggaagagcagattgcttatgccatgcagatgtccctgcagggagcagagtttggccaggcggaatcagcagacattgatgccagctcagctatggacacatctgagccagccaaggaggaggatgattacgacgtgatgcaggaccccgagttccttcagagtgtcctagagaacctcccaggtgtggatcccaacaatgaagccattcgaaatgctatgggctccctggcctcccaggccaccaaggacggcaagaaggacaagaaggaggaagacaagaagtgagactggagggaaagggtagctgagtctgcttaggggactgcatgggaagcacggaatatagggttagatgtgtgttatctgtaaccattacagcctaaataaagcttggcaactttttttccttttttgcttcaaa

SEQ ID NO: 24 - DNA, 2990 bp, Homo sapiens
tgcgcgcgcagcgggccggcagtggcggcggagatggaggagggaggccgggacaaggcgccggtgcagccccagcagtctccagcggcggcccccggcggcacggacgagaagccgagcggcaaggagcggcgggatgccggggacaaggacaaagaacaggagctgtctgaagaggataaacagcttcaagatgaactggagatgctcgtggaacgactaggggagaaggatacatccctgtatcgaccagcgctggaggaattgcgaaggcagattcgttcttctacaacttccatgacttcagtgcccaagcctctcaaatttctgcgtccacactatggcaaactgaaggaaatctatgagaacatggcccctggggagaataagcgttttgctgctgacatcatctccgttttggccatgaccatgagtggggagcgtgagtgcctcaagtatcggctagtgggctcccaggaggaattggcatcatggggtcatgagtatgtcaggcatctggcaggagaagtggctaaggagtggcaggagctggatgacgcagagaaggtccagcgggagcctctgctcactctggtgaaggaaatcgtcccctataacatggcccacaatgcagagcatgaggcttgcgacctgcttatggaaattgagcaggtggacatgctggagaaggacattgatgaaaatgcatatgcaaaggtctgcctttatctcaccagttgtgtgaattacgtgcctgagcctgagaactcagccctactgcgttgtgccctgggtgtgttccgaaagtttagccgcttccctgaagctctgagattggcattgatgctcaatgacatggagttggtagaagacatcttcacctcctgcaaggatgtggtagtacagaaacagatggcattcatgctaggccggcatggggtgttcctggagctgagtgaagatgtcgaggagtatgaggacctgacagagatcatgtccaatgtacagctcaacagcaacttcttggccttagctcgggagctggacatcatggagcccaaggtgcctgatgacatctacaaaacccacctagagaacaacaggtttgggggcagtggctctcaggtggactctgcccgcatgaacctggcctcctcttttgtgaatggctttgtgaatgcagcttttggccaagacaagctgctaacagatgatggcaacaaatggctttacaagaacaaggaccacggaatgttgagtgcagctgcatctcttgggatgattctgctgtgggatgtggatggtggcctcacccagattgacaagtacctgtactcctctgaggactacattaagtcaggagctcttcttgcctgtggcatagtgaactctggggtccggaatgagtgtgaccctgctctggcactgctctcagactatgttctccacaacagcaacaccatgagacttggttccatctttgggctaggcttggcttatgctggctcaaatcgtgaagatgtcctaacactgctgctgcctgtgatgggagattcaaagtccagcatggaggtggcaggtgtcacagctttagcctgtggaatgatagcagtagggtcctgcaatggagatgtaacttccactatccttcagaccatcatggagaagtcagagactgagctcaaggatacttatgctcgttggcttcctcttggactgggtctcaaccacctggggaagggtgaggccatcgaggcaatcctggctgcactggaggttgtgtcagagccattccgcagttttgccaacacactggtggatgtgtgtgcatatgcaggctctgggaatgtgctgaaggtgcagcagctgctccacatttgtagcgaacactttgactccaaagagaaggaggaagacaaagacaagaaggaaaagaaagacaaggacaagaaggaagcccctgctgacatgggagcacatcagggagtggctgttctggggattgcccttattgctatgggggaggagattggtgcagagatggcattacgaacctttggccacttgctgagatatggggagcctacactccggagggctgtacctttagcactggccctcatctctgtttcaaatccacgactcaacatcctggataccctaagcaaattctctcatgatgctgatccagaagtttcctataactccatttttgccatgggcatggtgggcagtggtaccaataatgcccgtctggctgcaatgctgcgccagttagctcaatatcatgccaaggacccaaacaacctcttcatggtgcgcttggcacagggcctgacacatttagggaagggcacccttaccctctgcccctaccacagcgaccggcagcttatgagccaggtggccgtggctggactgctcactgtgcttgtctctttcctggatgttcgaaacattattctaggcaaatcacactatgtattgtatgggctggtggctgccatgcagccccgaatgctggttacgtttgatgaggagctgcggccattgccagtgtctgtccgtgtgggccaggcagtggatgtggtgggccaggctggcaagccgaagactatcacagggttccagacgcatacaaccccagtgttgttggcccacggggaacgggcagaattggccactgaggagtttcttcctgttacccccattctggaaggttttgttatccttcggaagaaccccaattatgatctctaagtgaccaccaggggctctgaactgcagctgatgttatcagcaggccatgcatcctgctgccaagggtggacacggctgcagacttctgggggaattgtcgcctcctgctcttttgttactgagtgagataaggttgttcaataaagacttttatccccaaggaaaaaaaaaaaaaaaaaaaaaaaaaaaaaaaaaaaaaaaaaaaaaaaaaaaaaaaaaaaaaaaaaaaa

SEQ ID NO: 25 - DNA, 2357 bp, Homo sapiens
cgagcttggctgcttctggggcctgtgtggccctgtgtgtcggaaagatggagcaagaagccgagcccgaggggcggccgcgacccctctgaccgagatcctgctgctttcgcagccaggagcaccgtccctccccggattagtgcgtacgagcgcccagtgccctggcccggagagtggaatgatccccgaggcccagggcgtcgtgcttccgcgcgccccgtgaaggaaactggggagtcttgagggacccccgactccaagcgcgaaaaccccggatggtgaggagcaggcaaatgtgcaataccaacatgtctgtacctactgatggtgctgtaaccacctcacagattccagcttcggaacaagagaccctggttagaccaaagccattgcttttgaagttattaaagtctgttggtgcacaaaaagacacttatactatgaaagaggttcttttttatcttggccagtatattatgactaaacgattatatgatgagaagcaacaacatattgtatattgttcaaatgatcttctaggagatttgtttggcgtgccaagcttctctgtgaaagagcacaggaaaatatataccatgatctacaggaacttggtagtagtcaatcagcaggaatcatcggactcaggtacatctgtgagtgagaacaggtgtcaccttgaaggtgggagtgatcaaaaggaccttgtacaagagcttcaggaagagaaaccttcatcttcacatttggtttctagaccatctacctcatctagaaggagagcaattagtgagacagaagaaaattcagatgaattatctggtgaacgacaaagaaaacgccacaaatctgatagtatttccctttcctttgatgaaagcctggctctgtgtgtaataagggagatatgttgtgaaagaagcagtagcagtgaatctacagggacgccatcgaatccggatcttgatgctggtgtaagtgaacattcaggtgattggttggatcaggattcagtttcagatcagtttagtgtagaatttgaagttgaatctctcgactcagaagattatagccttagtgaagaaggacaagaactctcagatgaagatgatgaggtatatcaagttactgtgtatcaggcaggggagagtgatacagattcatttgaagaagatcctgaaatttccttagctgactattggaaatgcacttcatgcaatgaaatgaatcccccccttccatcacattgcaacagatgttgggcccttcgtgagaattggcttcctgaagataaagggaaagataaaggggaaatctctgagaaagccaaactggaaaactcaacacaagctgaagagggctttgatgttcctgattgtaaaaaaactatagtgaatgattccagagagtcatgtgttgaggaaaatgatgataaaattacacaagcttcacaatcacaagaaagtgaagactattctcagccatcaacttctagtagcattatttatagcagccaagaagatgtgaaagagtttgaaagggaagaaacccaagacaaagaagagagtgtggaatctagtttgccccttaatgccattgaaccttgtgtgatttgtcaaggtcgacctaaaaatggttgcattgtccatggcaaaacaggacatcttatggcctgctttacatgtgcaaagaagctaaagaaaaggaataagccctgcccagtatgtagacaaccaattcaaatgattgtgctaacttatttcccctagttgacctgtctataagagaattatatatttctaactatataaccctaggaatttagacaacctgaaatttattcacatatatcaaagtgagaaaatgcctcaattcacatagatttcttctctttagtataattgacctactttggtagtggaatagtgaatacttactataatttgacttgaatatgtagctcatcctttacaccaactcctaattttaaataatttctactctgtcttaaatgagaagtacttggtttttttttttcttaaatatgtatatgacatttaaatgtaacttattattttttttgagaccgagtcttgctctgttacccaggctggagtgcagtggcgtgatcttggctcactgcaagctctgcctcccgggttcgcaccattctcctgcctcagcctcccaattagcttggcctacagtcatctgccaccacacctggctaattttttgtacttttagtagagacagggtttcaccgtgttagccaggatggtctcgatctcctgacctcgtgatccgcccacctcggcctcccaaagtgctgggattacaggcatgagccaccg

SEQ ID NO: 26 - DNA, 1586 bp, Homo sapiens
agtggtggaggaacttccggcagcggcagctcaagtggccaaggcaagatgggtcaaagtcagagtggtggtcatggtcctggaggtggcaagaaggatgacaaggacaagaaaaagaaatatgaacctcctgtaccaactagagtggggaaaaagaagaagaaaacaaagggaccagatgctgccagcaaactgccactggtgacacctcacactcagtgccggttaaaattactgaagttagagagaattaaagactatcttctcatggaggaagaattcattagaaatcaggaacaaatgaaaccattagaagaaaagcaagaggaggaaagatcaaaagtggatgatctgagggggaccccgatgtcagtaggaaccttggaagagatcattgatgacaatcatgccatcgtgtctacatctgtgggctcagaacactacgtcagcattctttcatttgtagacaaggatctgctggaacctggctgctcggtcctgctcaaccacaaggtgcatgccgtgataggggtgctgatggatgacacggatcccctggtcacagtgatgaaggtagaaaaggccccccaggagacctatgcagatattggggggttggacaaccaaattcaggaaattaaggaatctgtggagcttcctctcacccatcctgaatattatgaagagatgggtataaagcctcctaagggggtcattctctatggtccacctggcacaggtaaaaccttgttagccaaagcagtagcaaaccaaacctcagccactttcttgagagtggttggctctgaacttattcagaagtacctaggtgatgggcccaaactcgtacgggaattgttccgagttgctgaagaacatgcaccgtccatcgtgtttattgatgaaattgacgccattgggacaaaaagatatgactccaattctggtggtgagagagaaattcagcgaacaatgttggaactgctgaaccagttggatggatttgattctaggggagatgtgaaagttatcatggccacaaaccgaatagaaactttggatccagcacttatcagaccaggccgcattgacaggaagattgagttccccctgcctgatgaaaagacgaagaagcgcatctttcagattcacacaagcaggatgacgctggctgatgatgtaaccctggacgacctgatcatggctaaagatgacctctctggtgctgacatcaaggcaatctgtacagaagctggtctgatggccttaagagaacgtagaatgaaagtaacaaatgaagacttcaaaaaatctaaagaaaatgttctttataagaaacaggaaggcacccctgaggggctgtatctctaatgaaccatggctgtcatcaggaaaatggttgggagatttctcaatccctgaaagggatgaggttgggggagttgcccagaggaatccctgttcccactgatttttattagcaaaacatcctgtgtcttttggagtacgatgtgtaagtgcccattgggtggcctgttggtcactgtgcagcagtctgcttcccaataaagcgtgctctttcacaa